(19) United States
(12) Patent Application Publication
(10) Pub. No.: US 2007/0207120 A1
(43) Pub. Date: Sep. 6, 2007

(54) Selective Killing Of Cancer Cells By Induction Of Acetyltransferase Via Tnf-Alpha And Il-6

(76) Inventors: Sarah Drayton, Cambridgeshire (GB); Yoshinori Takei, Cambridgeshire (GB)

Correspondence Address: KLARQUIST SPARKMAN, LLP, 121 SW SALMON STREET, SUITE 1600, PORTLAND, OR, 97204, US

(21) Appl. No.: 11/578,533

(22) PCT Filed: Apr. 13, 2005
(86) PCT No.: PCT/GB05/01408

§ 371 Date: Apr. 25, 2007

Related U.S. Application Data
(60) Provisional application No. 60/561,881, filed on Apr. 14, 2004.
(60) Provisional application No. 60/634,268, filed on Dec. 9, 2004.

(51) Int. Cl. A61K 48/00; A61K 38/19; A61K 39/395

(52) U.S. Cl. 424/85.1; 514/44; 424/145.1

(57) ABSTRACT

This invention relates to up-regulation of the acetyltransferase MCM3AP by the administration of the cytokines TNFalpha and IL-6 and, optionally a deacetylase inhibitor. This up-regulation is shown herein to be selectively lethal to cancer cells and has applications in the treatment of cancer.

This invention relates to the induction of cellular lethality in cancer cells through the up-regulation of cellular acetyltransferases.

MCM3 acetylating protein (MCM3AP) binds to and acetylates minichromosome maintenance protein 3 (MCM3) (1). MCM3 is one of the six MCM proteins that form the MCM complex that regulates DNA replication in eukaryotic cells. MCM3AP modifies MCM3 by acetylation and inhibits the initiation, but not the elongation, of DNA replication in vitro (2, 3). MCM3AP overexpression also inhibits entry into S-phase by transfected human cells (2, 3). Inhibition of entry into S-phase and inhibition of DNA replication in vitro depend on MCM3AP acetyltransferase activity and ability to bind to MCM3 (2, 3). Mutations in MCM3AP that abolish either acetyltransferase activity or ability to bind to MCM3 relieve the inhibition of DNA replication and S-phase entry in both assays.

Unusually, the gene encoding MCM3AP is contained entirely within the 3′ DNA sequence of a gene encoding a larger protein, called germinal centre associated nuclear protein, or GNP (4). This observation has led to the proposition that the two proteins are likely to be splice variants of each other, in which two different messenger RNAs arise by differential splicing from RNAs synthesised from the same gene promoter, upstream of the GANP gene (3-6). This proposition has been propagated in sequence databases in which the two genes have been categorised as splice variants of one another, leading to confusion in the literature. Thus GANP is sometimes erroneously referred to as MCM3AP (or Map80). The present inventors have now discovered that MCM3AP has its own promoter and is not a splice variant of GANP. The inventors have also recognised that certain combinations of cytokines act synergistically to up regulate the expression of MCM3AP without affecting GANP expression. Cytokine-induced up-regulation of MCM3AP is shown herein to selectively induce cell death in cancer cells but not normal cells.

One aspect of the invention provides a method of selectively killing a cancer cell comprising;
increasing or up-regulating the expression and/or activity of MCM3AP in said cell.

Up-regulation of MCM3AP expression is shown herein to have a selectively lethal effect on cancer cells but not normal non-cancer cells.

Methods of the invention encompass in vitro methods, in which the cancer cell is an isolated or cultured cell and in vivo methods in which the cancer cell is located within an individual having a cancer condition. Methods of the invention may be particularly useful, for example in the treatment of cancer and related conditions.

A method of treating cancer in an individual may comprise;
increasing or up-regulating the expression and/or activity of MCM3AP in one or more cancer cells in said individual.

The amino acid sequence of human MCM3AP has the database accession number BAA25170.1 (GI:2967446) and is also found in NP_{—}003897.2 (GI: 19923191). The nucleic acid sequence encoding human MCM3AP has the database accession number AB005543.1 (GI:2967445) and is also found in NM_{—}003906.3 (GI: 33469915) (Takei et al (1998) J. Biol. Chem. 273 (35), 22177-22180).

In preferred embodiments, the expression of MCM3AP may be increased or up-regulated in a cell by increasing the level or activity of both tumour necrosis factor α (TNFα) associated transcription factors and interleukin-6 (IL-6) associated transcription factors within a cell.

TNFα-associated transcription factors are polypeptides which are activated and/or up regulated in response to TNFα cytokine and which are active in modulating DNA transcription. TNFα associated transcription factors include NF-κB and AP-1.

The level and/or activity of TNFα associated transcription factor, such as NF-κB and/or AP-1, may be increased in a cell by exposing the cell to increased amounts of a TNFα cytokine, by increasing the activity of a TNFα cytokine to which the cell is exposed and/or by exposing the cell to an analogue or mimetic which possesses TNFα activity i.e. which increases the level or amount of TNFα associated transcription factors in a cell.

A cell may be exposed to increased amounts of a TNFα cytokine, for example, by contacting the cell with exogenous TNFα cytokine i.e. cytokine from a source which is not the cell, tissue or individual to which it is administered, contacted or exposed.

In some embodiments, a cell may be contacted with or exposed to recombinant cells which have been engineered to produce and secrete TNFα cytokines. Suitable recombinant cells may, for example, comprise a heterologous nucleic acid which encodes a TNFα cytokine and appropriate regulatory elements for directing the expression of the encoding nucleic acid. Alternatively, recombinant cells may be engineered to increase the expression and secretion of endogenous TNFα cytokines. In other embodiments, a cell with naturally high levels of TNFα expression and secretion may employed.

In some embodiments, a cell may be exposed to increased amounts of a TNFα cytokine by exposing the cell to cells in which expression and secretion of endogenous TNFα cytokine has been induced by contact with a TNFα cytokine inducer. This may be achieved for example, by administering a TNFα cytokine inducer to an individual.

Examples of TNFα inducers include LiCl (Beyaert et al PNAS USA (1989) 86(23) 9494-9498, Beyaert et al Cytokine (1991) 3 (4) 284-291), staurosporine (Beyaert et al Cancer Res (1993) 53 (11) 2623-2630), and phospholipase D (Beyaert et al Eur. J. Biochem. (1993) 212(2) 491-497).

The activity of a TNFα cytokine on a cell may be increased by contacting a cell with a TNFα enhancer or potentiator to the cell. Suitable enhancers include antibodies which specifically bind to TNFα receptors, in particular TNFα receptor II.

IL-6 associated transcription factors are polypeptides which are activated and/or up regulated in response to IL-6 cytokine and which are active in modulating DNA transcription. IL-6 associated transcription factors include NF-IL-6.

The level and/or activity of an IL-6 associated transcription factors, such as NF-IL-6, may be increased in a cell by exposing the cell to increased amounts of a IL-6 cytokine, by increasing the activity of a IL-6 cytokine to which the cell is exposed and/or by exposing the cell to an analogue or mimetic which possesses IL-6 activity i.e. which increases the level or amount of IL-6 associated transcription factors in a cell.

A cell may be exposed to increased amounts of an IL-6 cytokine, for example, by contacting the cell with exogenous IL-6 cytokine.

In some embodiments, a cell may be exposed to recombinant cells which have been engineered to produce and secrete IL-6 cytokines. Suitable recombinant cells may, for example, comprise a heterologous nucleic acid which encodes a IL-6 cytokine and appropriate regulatory elements for directing the expression of the encoding nucleic acid. Alternatively, recombinant cells may be engineered to increase the expression and secretion of endogenous IL-6 cytokines. In other embodiments, a cell with naturally high levels of IL-6 expression and secretion may employed.

In some embodiments, a cell may be exposed to increased amounts of an IL-6 cytokine by exposure to cells in which expression and/or secretion of endogenous IL-6 cytokine have been stimulated or enhanced by contact with an IL-6 cytokine inducer. Molecules which induce expression of IL-6 include IL-1 (Katz Y. et al. Arthritis Rheum 2001 44(9) 2176-84), IL-17 (Hwang S Y et al. Arthritis Res. Ther. 2004 6(2) R120-8 (Epub)), STAT3 (Signal Transducer and Activator of transcription 3), glucocorticoids, (Zhang Z et al. J Biol Chem 1997 272(49) 30607-10), Gfi-1 (Rodel B et al. 2000 EMBO Journal 19 5845-5855), IFNγ (Biondillo D E et al. Am J Physiol Lung Cell Mol Physiol 1994 267 L564-L568), cyclosporine A (Borger P et al. Abstract W.3 Workshop W, Mucosal Immunity), synthetic CpG oligodeoxynucleotides (Hasegawa K and Hayashi T. Lupus 2003 12(11) 838-845(8)) the herbal medicine shosaiko (Ohtake N et al. Int Immunopharmacol 2002 2(2-3) 357-66) and HIV-1 tat protein (Hofman F M et al Journal of Neuroimmunology 1999 94 28-39).

The activity of an IL-6 cytokine on a cell may be increased by contacting a cell with an IL-6 enhancer or potentiator to the cell. IL-6 potentiators may include antibodies which specifically bind to IL-6. Suitable antibodies are commercially available (e.g. Abcam Ltd, Cambridge UK).

A cancer cell within an individual having a cancer condition may be contacted with or exposed to a cytokine, analogue or mimetic as described above, by administering the cytokine, or a cell expressing the cytokine, or an analogue or mimetic of the cytokine or a cytokine inducer or potentiator to the individual. A method of treating a cancer condition in an individual may comprise administering to said individual;

one or more of a TNFα cytokine, a nucleic acid encoding a TNFα cytokine, a recombinant cell expressing a TNFα cytokine, a TNFα potentiator, a TNFα inducer and a TNFα analogue or mimetic, and;

one or more of: a IL-6 cytokine, a nucleic acid encoding an IL-6 cytokine, a recombinant cell expressing an IL-6 cytokine, an IL-6 potentiator, an IL-6 inducer and an IL-6 analogue or mimetic.

A TNFα cytokine may include a human polypeptide having the sequence of NP_{—}000585.2 (GI:25952111) or a fragment, allele or variant thereof.

An IL-6 cytokine may include a human polypeptide having the sequence of NP_{—}000591.1 (GI:10834984) or a fragment, allele or variant thereof.

An allele or variant may have an amino acid sequence which differs from a given cytokine sequence, by one or more of addition, substitution, deletion and insertion of one or more amino acids but which still has substantially the same sequence as the given sequence. Such an addition, substitution, deletion or insertion may represent a natural variation which occurs between individuals within a species and which has no phenotypic effect. A fragment, allele or variant of a cytokine retains some or all of the biological activity of the parent cytokine, in particular the activation of transcription factors.

A cytokine which is an amino acid sequence variant or allele may comprise an amino acid sequence which differs from a given cytokine amino acid sequence, but which shares greater than about 50% sequence identity with such a sequence, greater than about 60%, greater than about 70%, greater than about 80%, greater than about 90% or greater than about 95%. A variant or allelic sequence may share greater than about 60% similarity, greater than about 70% similarity, greater than about 80% similarity or greater than about 90% similarity with a given cytokine amino acid sequence.

A cytokine which is a fragment of the full-length sequence may consist of at least 25 amino acids, at least 50 amino acids, at least 100 amino acids, at least 150 amino acids or at least 200 amino acids of the full-length amino acid sequence.

IL-6 and TNFα cytokines suitable for use in methods described herein are readily available from commercial sources. Alternatively, nucleic acid sequences which encode and express IL-6 and TNFα cytokines may be readily prepared by the skilled person using the information and references contained herein and techniques known in the art (for example, see Sambrook, Fritsch and Maniatis, Molecular Cloning, A Laboratory Manual, Cold Spring Harbor Laboratory Press, 1989, and Ausubel et al, Short Protocols in Molecular Biology, John Wiley and Sons, 1992), given the nucleic acid sequences which are publicly available. These techniques include (i) the use of the polymerase chain reaction (PCR) to amplify samples of such nucleic acid, e.g. from genomic sources, (ii) chemical synthesis, or (iii) preparing cDNA sequences.

For expression of nucleic acid coding sequences, the sequences can be incorporated in a vector having one or more control sequences operably linked to the nucleic acid to control its expression. The vectors may include other sequences such as promoters or enhancers to drive the expression of the inserted nucleic acid, nucleic acid sequences so that the polypeptide or peptide is produced as a fusion and/or nucleic acid encoding secretion signals so that the polypeptide produced in the host cell is secreted from the cell. Polypeptide can then be obtained by transforming the vectors into host cells in which the vector is functional, culturing the host cells so that the polypeptide is produced and recovering the polypeptide from the host cells or the surrounding medium.

Systems for cloning and expression of a polypeptide, such as a cytokine, in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

In some embodiments, as described above, a cytokine may be produced in situ from an encoding nucleic acid. For example, the encoding nucleic acid or a cell comprising the nucleic acid may be administered to an individual having a cancer condition. Preferably, the coding sequence is operably linked to a regulatory element suitable for expression in a host cell. The coding sequence may be contained in an expression vector, for example a virus or plasmid vector. The vector may be administered to an individual for transformation or transfection of cells in vivo or may be used to transform or transfect cells in vitro, prior to administration of the transformed cells to an individual. Suitable techniques and protocols for performing such methods are well known in the art.

In some embodiments, an IL-6 and/or TNFα analogue or mimetic may be used to increase the levels or activity of IL-6 and/or TNFα associated transcription factors, respectively, within a cell. For example, a TNFα analogue or mimetic may be used to activate the transcription factors NF-κB and AP-1 and an IL-6 analogue or mimetic may be used to activate transcription factor NF-IL-6.

Suitable analogues include chemical compounds which are modelled using rational drug design to resemble the three dimensional structure of all or part of the cytokine, and in particular the arrangement of the key amino acid residues.

Suitable modelling techniques are known in the art. This includes the design of so-called “mimetics” which involves the study of the functional interactions of the molecules and the design of compounds which contain functional groups arranged in such a manner that they could reproduced those interactions.

The preparation of analogues and mimetics based on a ‘lead’ compound identified as biologically active, for example a cytokine, is a known approach to the development of pharmaceuticals and may be desirable where the active compound is difficult or expensive to synthesise or where it is unsuitable for a particular method of administration, e.g. cytokines and other peptides are not well suited as active agents for oral compositions as they tend to be quickly degraded by proteases in the alimentary canal. Modification of a known active cytokine (for example, to produce a mimetic) may be used to avoid randomly screening large number of compounds for a target property.

Modification of a ‘lead’ compound, such as a cytokine, to optimise its pharmaceutical properties commonly comprises several steps. Firstly, the particular parts of the compound that are critical and/or important in determining the target property are determined. In the case of a cytokine or other peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

A template molecule is then selected onto which chemical groups that mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have cytokine activity, or to what extent they exhibit it. Modified compounds include cytokine analogues and mimetics.

Further optimisation or modification can then be carried out to arrive at one or more final cytokine analogues or mimetics for in vivo or clinical testing.

In preferred embodiments, a cell may be exposed to or contacted with an IL-6 cytokine and a TNFα cytokine in the presence of an anti-TNFα receptor antibody.

An anti-TNFα receptor antibody is an antibody which binds specifically to an anti-TNFα receptor such as anti-TNFα receptor II.

An antibody which binds specifically to a target epitope does not show any significant binding to molecules which do not contain the target epitope. If an antibody is specific for a particular epitope which is carried by a number of antigens, the antibody will be able to bind to the various antigens carrying the epitope.

In some preferred embodiments, a method of treating a cancer condition in an individual may comprise administering a combination of an IL-6 cytokine and a TNFα cytokine to the individual, and optionally further administering a TNFα receptor-specific antibody to the individual.

The terms ‘TNFα factor’ and ‘IL-6 factor’ are used herein to denote agents which may be used to increase the level and/or activity of TNFα and IL-6 associated intracellular signalling molecules in a cell, respectively. Examples of suitable TNFα factors and IL-6 factors include TNFα and IL-6 cytokines and inducers, potentiators, analogues and mimetics thereof, as well as nucleic acid encoding TNFα and IL-6 cytokines and cells expressing TNFα and IL-6 cytokines.

A method as described herein may comprise administering more than one, for example two or more TNFα factors and more than one, for example two or more IL-6 factors.

The experiments described herein show that deacetylase inhibitors have a synergistic effect on the cytokine treatment described above.

A method of selectively killing a cancer cell in an individual as described herein may further comprise contacting the cancer cell with a deacetylase inhibitor.

The cancer cell may be contacted with the deacetylase inhibitor by administering the deacetylase inhibitor to the individual.

A deacetylase inhibitor is a compound which reduces or inhibits protein deacetylation in a cell, in particular the deacetylation of MCM3 in a cell.

Many suitable deacetylase inhibitors are known in the art, including, for example, hydroxamates, cyclic peptides, aliphatic acids, benzamides and electrophilic ketones.

Hydroxamates include trichostatin A (TSA), suberoylanilide hydroxamic acid (SAHA), cinnamic acid bishydroxamic acid (aka M-carboxycinnamic acid bishydroxamide) (CBHA), RO4474861 (Roche), hexamethylenebisacetamide (HMBA), diethyl bis-(pentamethylene-N, N-dimethylcarboxamide) malonate (EMBA), LAQ-824 (aka NVP-LAQ-824) (Novartis), Sulfonamide hydroxamic acids, pyroxamide, oxamflatin, scriptaid (SB-556629: GSK), 4-pyridin-4-yl-1,3-imidazole-2-thione (SB-379872-A: GSK) and PXD-101. Hydroxamates are preferred deacetylase inhibitors in some embodiments of the invention.

Cyclic peptides include cyclic tetrapeptides such as depsipeptide (FK-228) (Fujisawa), apidicin, spiruchostatin A, epi-spiruchostatin A, trapoxin (TPX), and TPX-HA analogues (e.g. CHAP—cyclic hydroxamic acid-containing peptide such as CHAP31 (Riken)).

Aliphatic acids include valproic acid, phenyl butyrate and butyric acid.

Benzamides include MS-275, CI-994 (N-acetylamide) and SB-499201 (N-(4-phenoxyphenyl)-4-(3-phenylpropanoylamino)-benzamide).

Electrophilic ketones include trifluoromethyl ketones and α-ketoamides.

Other suitable deacetylase inhibitors are described in Marks, P. A et al (2003) Current Opinion in Pharmacology 3:344-351; Richon V. M. et al (1996) PNAS 93:5705-5708; Villar-Garea A. et al (2004) Int. J. Cancer 112:171-178; McLaughlin F′ et al (2004) Biochemical Pharmacology 68:1139-1144; Fuino et al Mol Cancer Ther. 2003 2(10) :971-84; Aron et al Blood 2003 102 652-658; Yurek-George et al J Am Chem Soc. 2004 126(4):1030-1; and Hu et al JPET 307:720-728, 2003.

In some embodiments, a deacetylase inhibitor which is not a butyric acid or is not an aliphatic acid may be preferred.

In other embodiments, a deacetylase inhibitor may be selected from the group consisting of CBHA, SAHA, TSA and butyric acid may be preferred.

Other aspects of the invention provide a combination of a TNFα factor, an IL-6 factor and, optionally a deacetylase inhibitor for use in a method of treatment of the human or animal body, for example for use in a method of treating cancer, and the use of a

TNFα factor, an IL-6 factor and, optionally, a deacetylase inhibitor, in the manufacture of a medicament for use in the treatment of cancer.

A suitable TNFα factor may include one or more of: a TNFα cytokine, a nucleic acid encoding a TNFα cytokine, a recombinant cell expressing a TNFα cytokine, a TNFα potentiator, a TNFα inducer and a TNFα analogue or mimetic.

A suitable IL-6 factor may include one or more of: a Il-6 cytokine, a nucleic acid encoding an IL-6 cytokine, a recombinant cell expressing an IL-6 cytokine, an IL-6 potentiator, an IL-6 inducer and an IL-6 analogue or mimetic.

Suitable deacetylase inhibitors are described above.

A cancer as described herein may include any type of solid cancer or malignant lymphoma and especially leukaemia, sarcomas, skin cancer, bladder cancer, breast cancer, uterus cancer, ovary cancer, prostate cancer, lung cancer, colorectal cancer, cervical cancer, liver cancer, head and neck cancer, oesophageal cancer, pancreas cancer, renal cancer, stomach cancer and cerebral cancer. In some preferred embodiments, the cancer condition may be breast, ovary, pancreas or prostate cancer. Cancers may be familial or sporadic.

In some preferred embodiments, over-expression of MCM3AP may be selectively lethal to cancer cells that are deficient in p53 expression and/or activity. Thus, a cancer as described herein may be a p53 deficient cancer i.e. a cancer in which the activity or expression of the tumour suppressor p53 is reduced or abrogated. The amino acid sequence of human p53 has the database accession number NP_{—}000537.2 and the gene identifier GI: 8400738. p53 expression or activity may be deficient in many different types of cancer. In some cancers, such as lung, colon, head/neck, ovary, bladder and non-melanoma skin cancers, there is a high frequency of p53 deficiency (e.g. >50%).

Over-expression of MCM3AP may also be selectively lethal to cancer cells that are deficient in expression and/or activity of the retinoblastoma susceptibility gene (Rb). Thus, a cancer as described herein may be an Rb deficient cancer i.e. a cancer in which the activity or expression of Rb or its downstream effectors is reduced or abrogated. The coding sequence of human Rb has the database accession number M28419 and the gene identifier 190962 and the amino acid sequence of pRb has the database accession number AAA69808.1. Rb expression or activity may be deficient in many different types of cancer.

Cancers deficient in p53 and/or Rb expression and/or activity may be identified using standard techniques. For example, p53 or Rb deficiency may be detected at the nucleic acid level by detecting mutations in the nucleic acid encoding p53 or Rb or the elements regulating its expression, or at the protein level by detecting the level of p53 or Rb expression and/or the presence of mutant polypeptide using specific antibodies. These techniques may be performed on a sample of cancer cells obtained from the individual. A method described herein may comprise identifying a cancer or cancer cell as deficient in p53 expression or activity and/or Rb expression or activity.

TNFα and IL-6 factors and, optionally, deacetylase inhibitors may be administered simultaneously or sequentially in the methods described herein.

While it is possible for the active compounds to be administered alone, it is preferable to present them as a pharmaceutical composition (e.g., formulation) comprising at least one active compound, as defined above, preferably two, and optionally three or more, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Other aspects of the invention provide a pharmaceutical composition comprising a TNFα factor, an IL-6 factor, a pharmaceutically acceptable excipient and, optionally, a deacetylase inhibitor and a method of making a pharmaceutical composition comprising admixing a TNFα factor, an IL-6 factor and, optionally, a deacetylase inhibitor as described herein with a pharmaceutically acceptable excipient.

Pharmaceutical compositions comprising cytokine factors as defined above, for example one or more factors admixed together with one or more pharmaceutically acceptable carriers, excipients, buffers, adjuvants, stabilisers, or other materials, as described herein, may be used in the methods described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The cytokine factors or pharmaceutical composition(s) comprising the cytokine factors, optionally in combination with a deactylase inhibitor, may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; an d aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer=s Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for a injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and rotate of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

The experiments described herein show that the up-regulation of MCM3AP provides a selective anti-cancer effect. Other aspects of the invention provide screening methods for compounds which up-regulate MCM3AP and may therefore have potential as anti-cancer therapeutics.

A method of identifying and/or obtaining an anti-cancer agent may comprise;
providing a nucleic acid construct comprising a MCM3AP promoter operably linked to a reporter gene,
contacting the construct with a test compound and;
determining the expression of the reporter gene.

The level of expression in the presence of the test compound may be compared with the level of expression in the absence of the test compound. A difference in expression in the presence of the test compound indicates ability of the compound to modulate MCM3AP expression, for example an increase in expression indicates that the compound up-regulates the expression of MCM3AP. An increase in expression of the reporter gene compared with expression of another gene not linked to a promoter as disclosed herein indicates that the test compound has specificity for the MCM3AP promoter. Such a compound may be useful in the treatment of cancer as described herein.

A promoter is a sequence of nucleotides from which transcription may be initiated of DNA operably linked downstream (i.e. in the 3′ direction on the sense strand of double-stranded DNA).

‘Operably linked’ means joined as part of the same nucleic acid molecule, suitably positioned and oriented for transcription to be initiated from the promoter. DNA operably linked to a promoter is “under transcriptional initiation regulation” of the promoter.

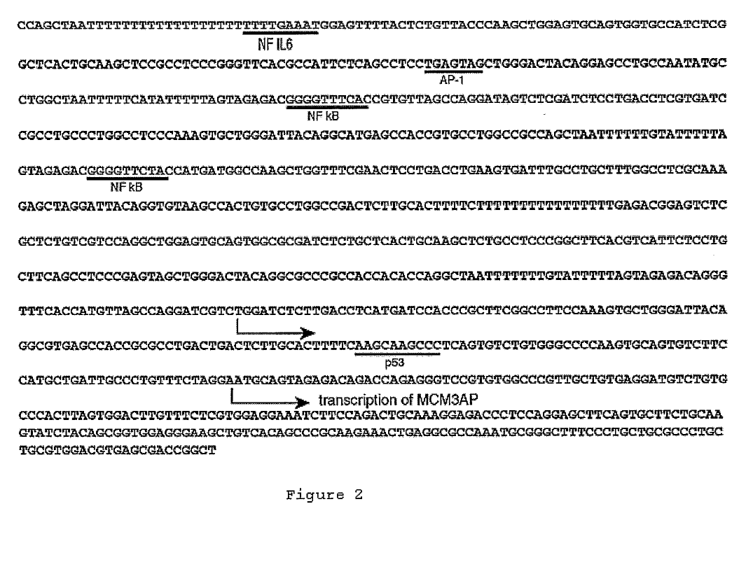

An MCM3AP promoter may comprise the full sequence shown in FIG. 2 or may be a variant or a fragment thereof, in particular a variant or a fragment which comprises one or more transcription factor binding sites indicated in FIG. 2 and retains some or all promoter activity. For example, the MCM3AP promoter may comprise the sequence of FIG. 2 upstream of the indicated MCM3AP transcription start site.

A fragment or variant of the MCM3AP promoter sequence may differ from the sequence of FIG. 2 by the addition, deletion, substitution and/or insertion of one or more amino acids, provided the promoter function is retained.

For example, an MCM3AP promoter may comprise an amino acid sequence which shares greater than about 30% sequence identity with FIG. 2, greater than about 40%, greater than about 45%, greater than about 55%, greater than about 65%, greater than about 70%, greater than about 80%, greater than about 90% or greater than about 95%. The sequence may share greater than about 30% similarity with FIG. 2, greater than about 40% similarity, greater than about 50% similarity, greater than about 60% similarity, greater than about 70% similarity, greater than about 80% similarity or greater than about 90% similarity.

Sequence similarity and identity are defined with reference to the algorithm GAP (Genetics Computer Group, Madison, Wis.). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Sequence identity and similarity may also be determined using Genomequest™ software (Gene-IT, Worcester Mass. USA).

Sequence comparisons are preferably made over the full-length of the relevant sequence described herein.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine.

The reporter gene preferably encodes an enzyme which catalyses a reaction which produces a detectable signal, preferably a visually detectable signal, such as a coloured product. Many examples are known, including GFP, luciferase, GUS, β-galactosidase, β-glucoronidase, and chloramphenicol acetyl transferase. β-galactosidase activity may be assayed, for example, by production of blue colour on substrate, the assay being by eye or by use of a spectro-photometer to measure absorbance. Fluorescence, for example that produced as a result of luciferase activity, may be quantitated using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labelled directly or indirectly using any standard technique.

Those skilled in the art are well aware of a multitude of possible reporter genes and assay techniques which may be used to determine gene activity.

A construct comprising the MCM3AP promoter operably linked to a reporter gene may be made using standard techniques. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds. John Wiley & Sons, 1992.

The nucleic acid construct may be contained in an expression vector which is, for example, introduced into a host cell by transformation or transfection techniques. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press.

A screening method of the invention may be an in vivo cell-based method which is performed in a cell line such as a yeast strain, insect or mammalian cell line, for example CHO, HeLa or COS cells. A nucleic acid construct may be introduced into a cell line using any technique previously described to produce a stable cell line containing the reporter construct integrated into the genome. The cells may be grown and incubated with test compounds for varying times. The test compound may thus be contacted with an expression system, such as a host cell, which contains the nucleic acid construct. The cells may be grown in 96 well plates to facilitate the analysis of large numbers of compounds. The cells may then be washed and the reporter gene expression analysed. For some reporters, such as luciferase the cells will be lysed then analysed.

In some embodiments, the construct may be contacted with the test compound in the presence of a TNFα or IL-6 cytokine.

The precise format for performing methods as described herein may be varied by those of skill in the art using routine skill and knowledge.

A suitable test compounds may be natural or synthetic chemical compounds used in drug screening programmes. Extracts of plants which contain several characterised or uncharacterised components may also be used. Combinatorial library technology (Schultz, J S (1996) Biotechnol. Prog. 12:729-743) provides an efficient way of testing a potentially vast number of different substances for ability to modulate activity of a polypeptide.

The amount of test substance or compound which may be added to a screen will normally be determined by trial and error depending upon the type of compound used. Typically, from about 0.1 to 100 μM concentrations of putative inhibitor compound may be used, for example from 1 to 10 μM. When cell-based methods are employed, the test substance or compound is desirably membrane permeable in order to access the nucleic acid construct.

Methods of the invention may comprise the step of identifying a test compound as an up-regulator of MCM3AP expression. The test compound may be further identified as an anti-cancer agent.

Following identification of such a compound, the compound may be investigated further. A method may comprise, for example, isolating and/or synthesising said test compound.

Following identification of a compound as described above, a method may further comprise modifying the compound to optimise the pharmaceutical properties thereof.

Further optimisation or modification can then be carried out to arrive at one or more final compounds for in vivo or clinical testing.

The test compound may be manufactured and/or used in preparation, i.e. manufacture or formulation, of a composition such as a medicament, pharmaceutical composition or drug. These may be administered to individuals, e.g. for any of the purposes discussed elsewhere herein.

Thus, the present invention extends in various aspects not only to a compound identified as disclosed herein, but also a pharmaceutical composition, medicament, drug or other composition comprising such a compound, a method comprising administration of such a composition to a patient, e.g. for increasing MCM3AP expression for instance in treatment of a cancer condition, use of such a compound in manufacture of a composition for administration, e.g. for increasing MCM3AP expression for instance in treatment of cancer and a method of making a pharmaceutical composition comprising admixing such a compound with a pharmaceutically acceptable excipient, vehicle or carrier, and optionally other ingredients.

Further aspects of the invention provide a nucleic acid construct comprising a MCM3AP promoter operably linked to a heterologous gene, an expression vector comprising such a construct and a host cell comprising such a construct or vector for use in a screening method as described above.

Constructs, vectors and cells are described in more detail above.

All documents mentioned in this specification are hereby incorporated herein by reference.

It will be understood that the invention encompasses each and every combination and sub-combination of the features of the invention described above.

Aspects of the present invention will now be illustrated with reference to the accompanying figures described below and experimental exemplification, by way of example and not limitation. Further aspects and embodiments will be apparent to those of ordinary skill in the art.

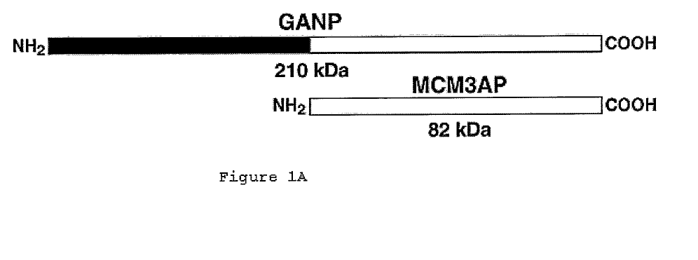

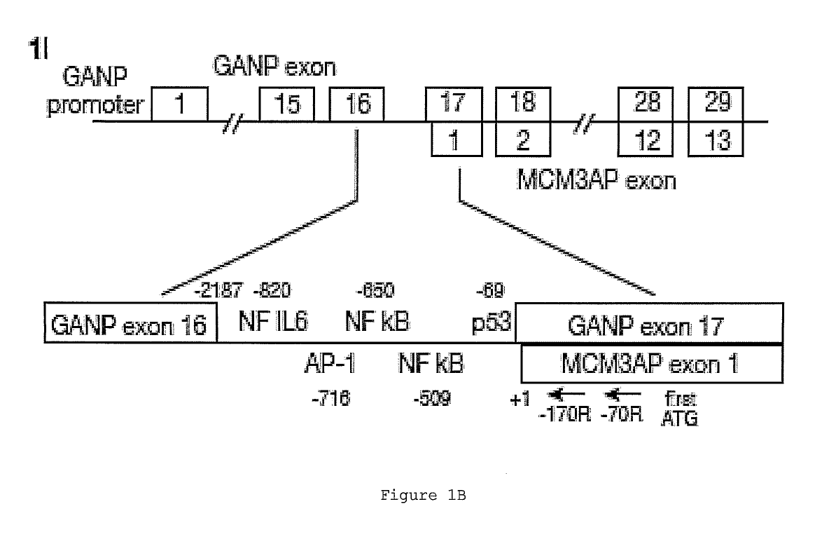

FIG. 1a shows that the 82-kilodalton MCM3AP protein is identical to the carboxy terminal region of GANP protein and is encoded in the 3 prime end of the GANP gene.

FIG. 1b shows that intron 16 (previously named intron 15) of GANP, which is the sequence upstream of exon 1 of MCM3AP, contains consensus sequence binding sites for transcription factors NF-IL6, AP1, NF-kappa B and p53. Initiation of transcription maps near the start of exon 1.

FIG. 2 shows sequence of intron 16 (previously named intron 15) of GANP, which is the MCM3AP promoter region. The consensus binding sites for NF-IL6, NF-kappa B, AP1 and p53, are indicated and also the sequence at which transcription initiates, as mapped by RACE (Rapid Amplification of cDNA Ends) and oligonucleotide capping.

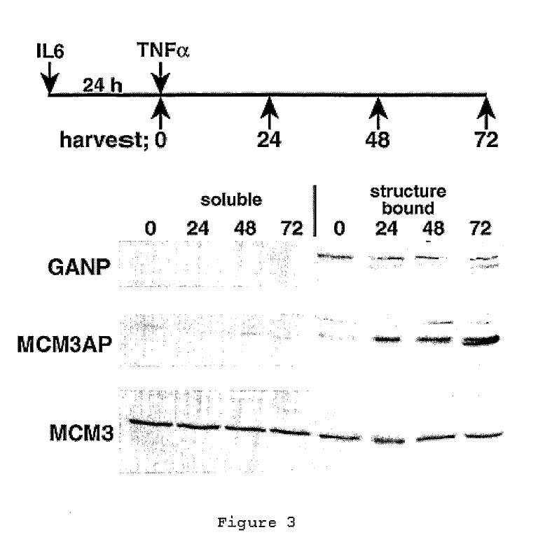

FIG. 3 shows the results of western blot analysis which indicates that MCM3AP, but not GANP, expression is induced in HEK 293 cells (ATCC number CRL-1573) by cytokine treatment.

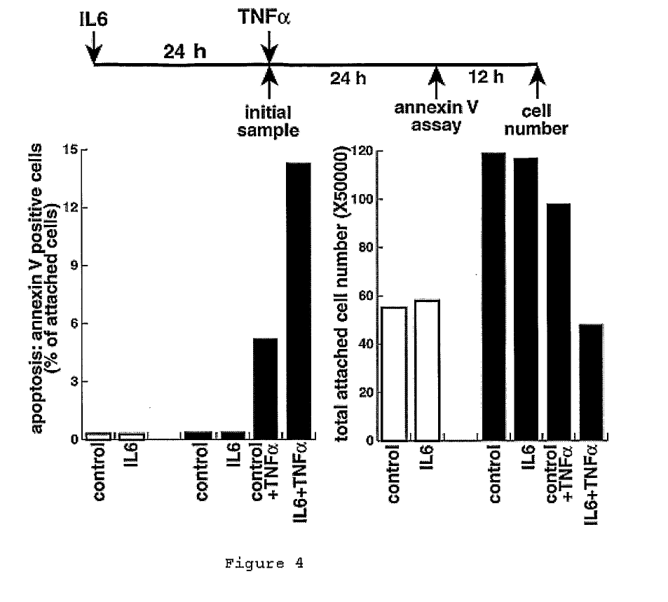

FIG. 4 shows that the same cytokine treatment that induces MCM3AP expression also activates apoptosis of HEK 293 cells. In the left hand histograms, apoptotic cell death is monitored by annexin-V staining showing extensive induction of dying cells by IL6 and TNFα. In the right hand histograms, the attached cell number is recorded showing that the combination of interleukin 6 and TNFα leads to a decrease in cell number rather than the marked increase seen in control cells.

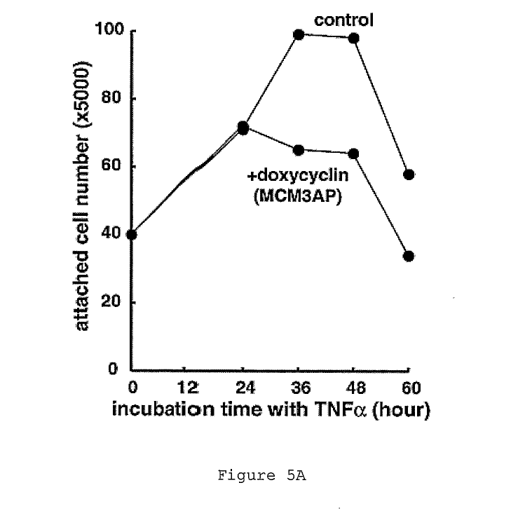

FIG. 5 shows that stable over-expression of MCM3AP in HEK 293 cells augments TNFα-induced apoptosis. FIG. 5A shows a reduction in cell numbers in the presence of doxycycline induced MCM3AP expression. FIG. 5B shows the % of annexin V and TUNEL positive cells at 0 hr and 24 hr after doxycycline induced MCM3AP expression.

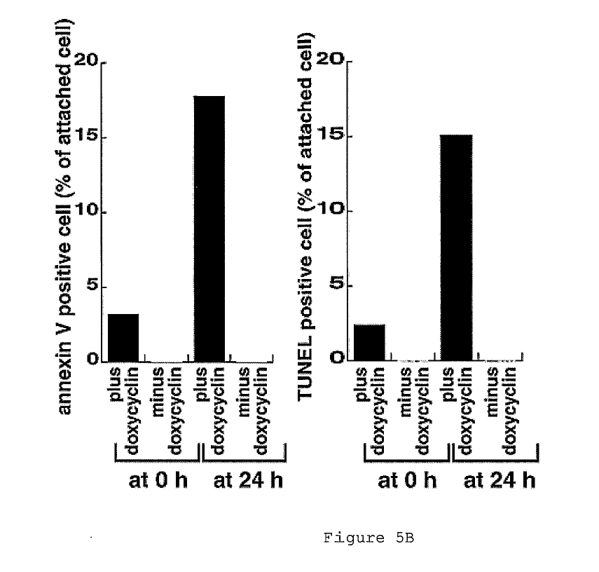

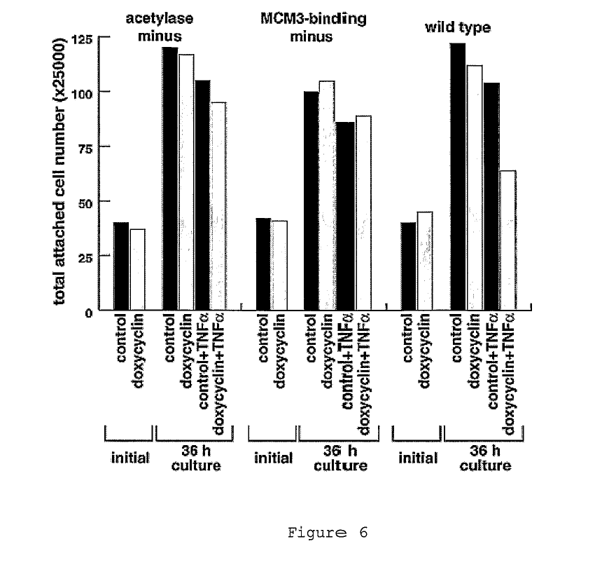

FIG. 6 shows that cytokine dependent killing of stably transfected HEK 293 cells is sensitized by wild type MCM3AP, but not by MCM3AP carrying mutations that abolish either acetyltransferase activity or MCM3 binding activity.

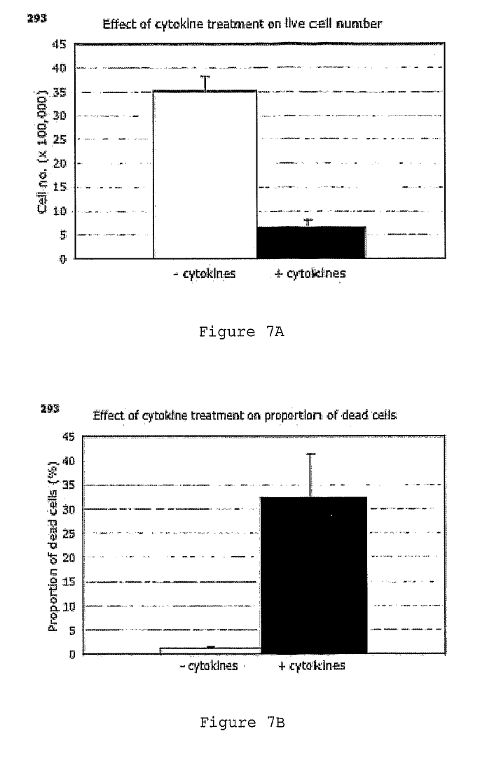

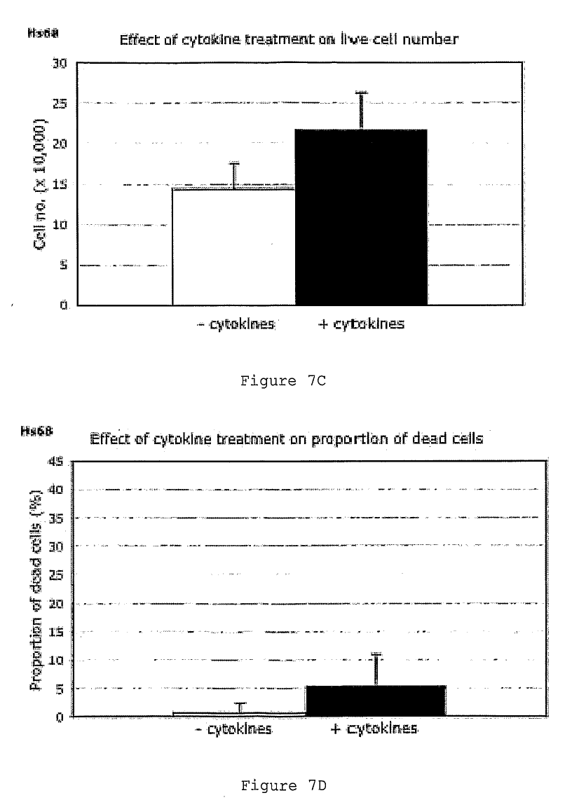

FIG. 7 shows that cytokine treatments that cause apoptotic cell death in HEK 293 cells do not kill normal human fibroblasts, Hs68 cells (ATCC number CRL-1635). FIG. 7A shows that cytokine treatment causes a marked decrease in live HEK293 cells. FIG. 7B shows that cytokine treatment causes a marked increase in the proportion of dead HEK293 cells. FIG. 7C shows that cytokine treatment does not cause a decrease in live Hs68 cells and FIG. 7D shows that cytokine treatment does not does not cause a significant increase in the proportion of dead Hs68 cells.

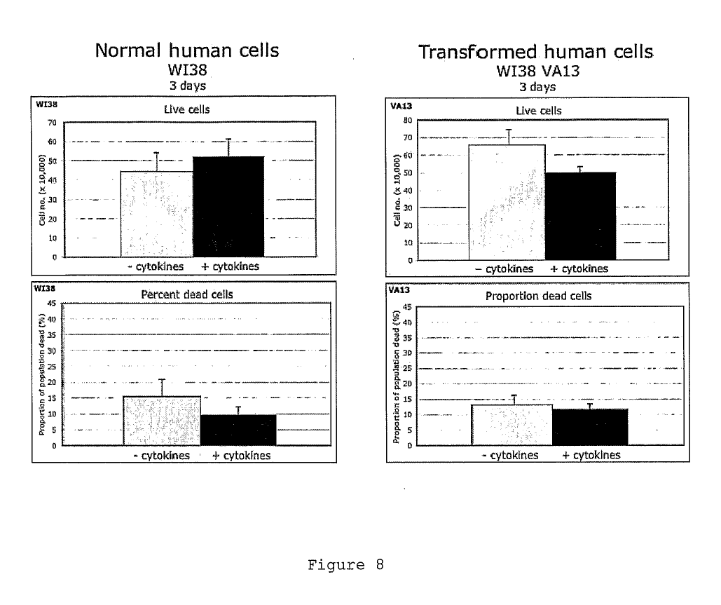

FIG. 8 shows the effect of cytokine treatment for 3 days on normal WI38 cells and transformed WI38 VA13 cells (see materials and methods) cells.

FIG. 9 shows the effect of cytokine treatment for 4 days on normal and transformed W138 cells.

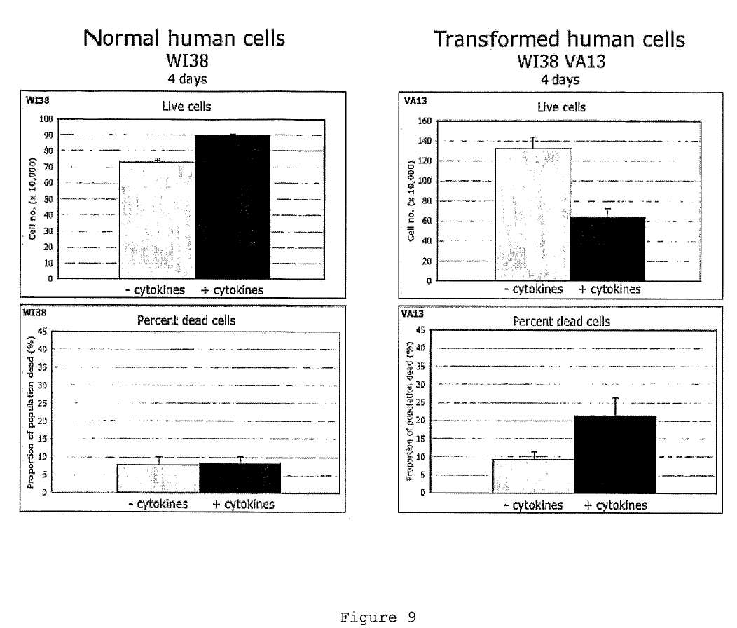

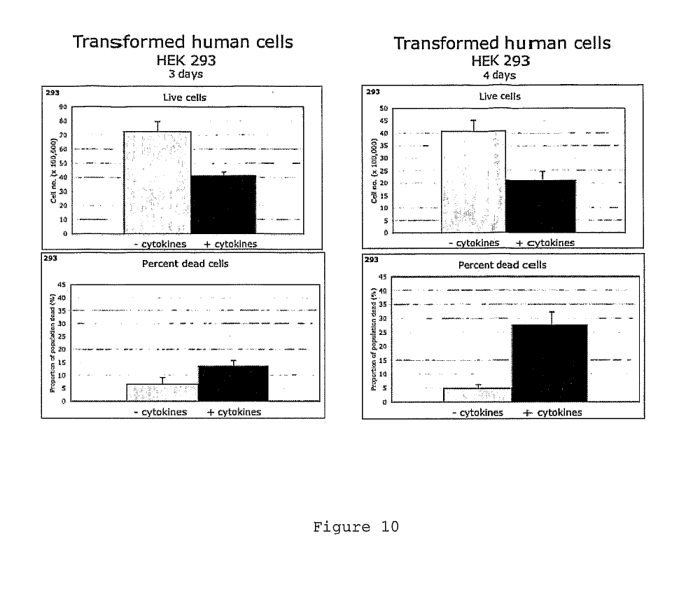

FIG. 10 shows the effect of cytokine treatment for 3 and 4 days on HEK293 cells.

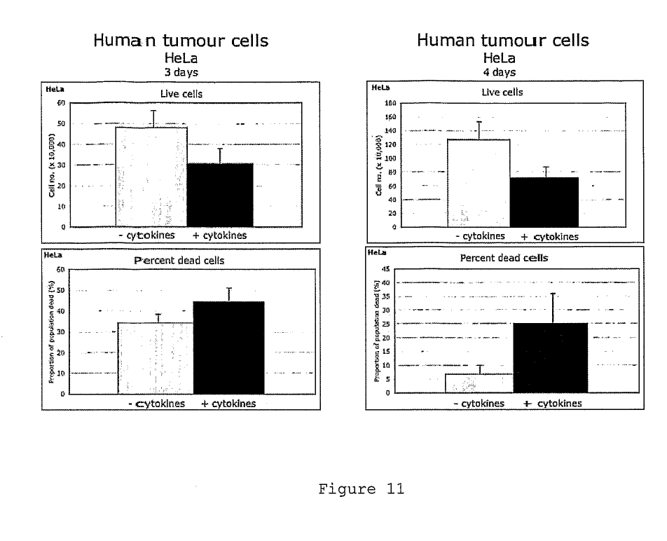

FIG. 11 shows the effect of cytokine treatment for 3 and 4 days on Hela cells.

FIG. 12 shows the effect of cytokine treatment for 4 days on U2OS cells.

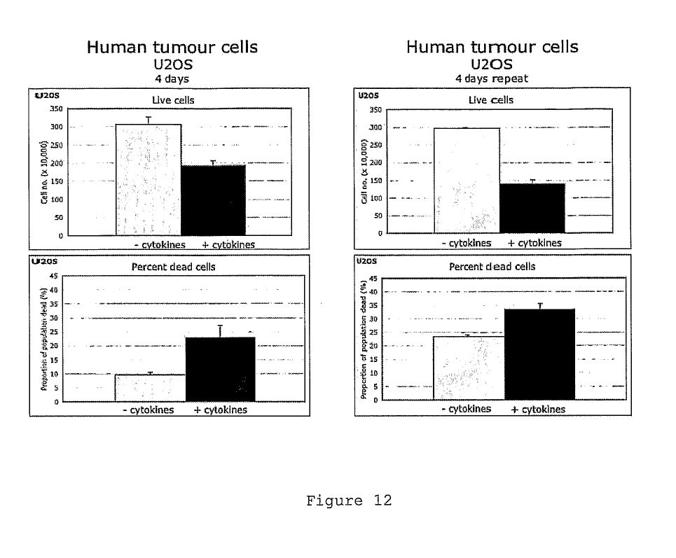

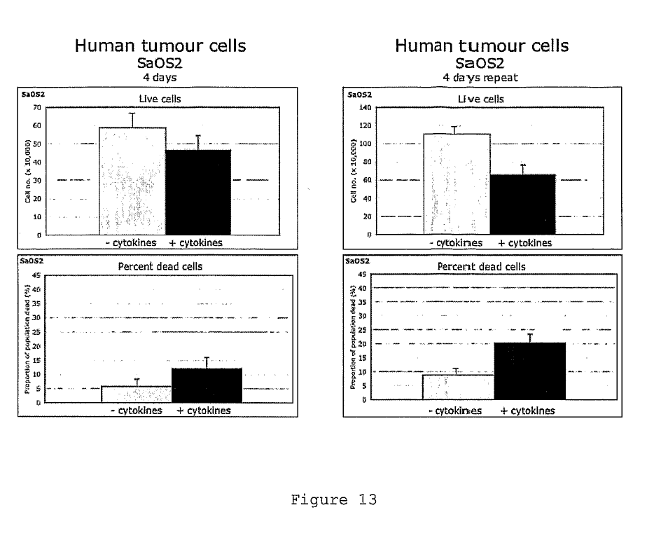

FIG. 13 shows the effect of cytokine treatment for 4 days on SaOS2 cells.

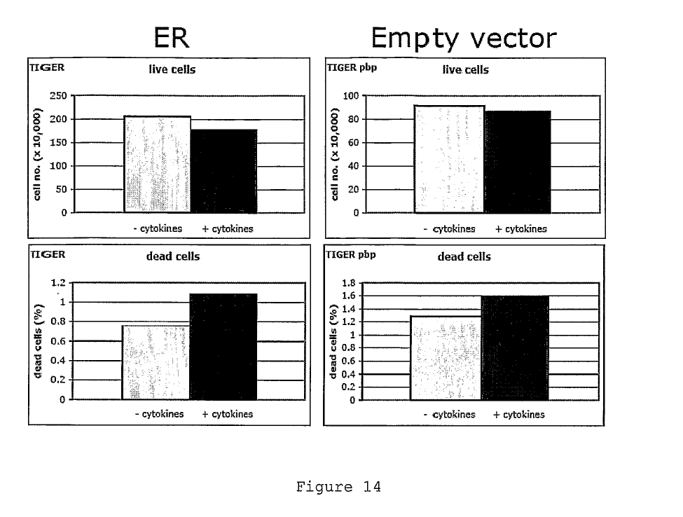

FIG. 14 shows the effect of cytokine treatment for 4 days on primary human fibroblasts (TIG-3) expressing the ecotropic receptor (ER) and TIG-3 cells expressing the empty pBABEpuro vector.

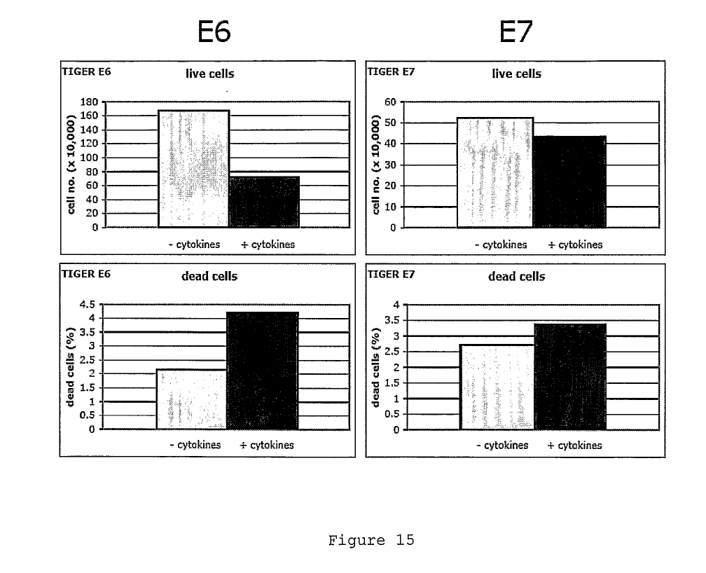

FIG. 15 shows the effect of cytokine treatment for 4 days on primary human fibroblasts (TIG-3) expressing the viral oncoproteins Human Papilloma Virus (HPV) E6 or Human Papilloma Virus (HPV) E7.

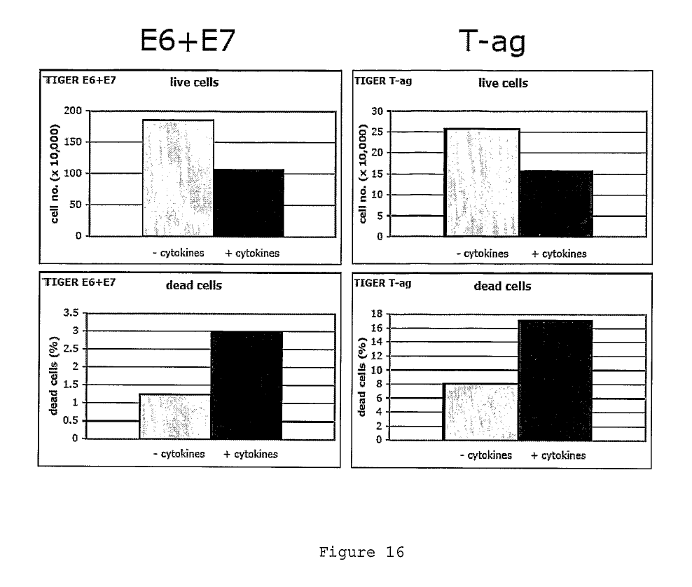

FIG. 16 shows the effect of cytokine treatment for 4 days on primary human fibroblasts (TIG-3) expressing both Human Papilloma Virus (HPV) E6 and E7 or expressing Simian Virus 40 (SV40) large T-antigen.

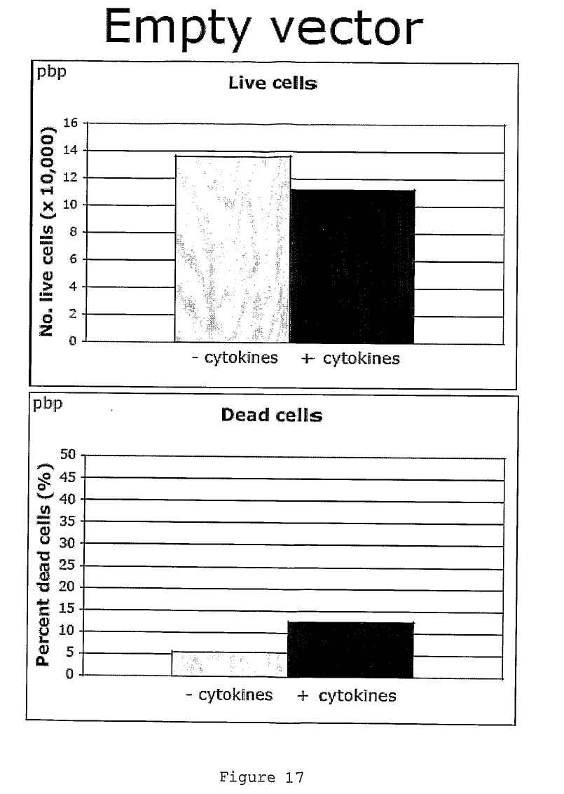

FIG. 17 shows the effect of cytokine treatment for 8 days on primary human fibroblasts (TIG-3) TIG-3 cells expressing the empty pBABEpuro vector.

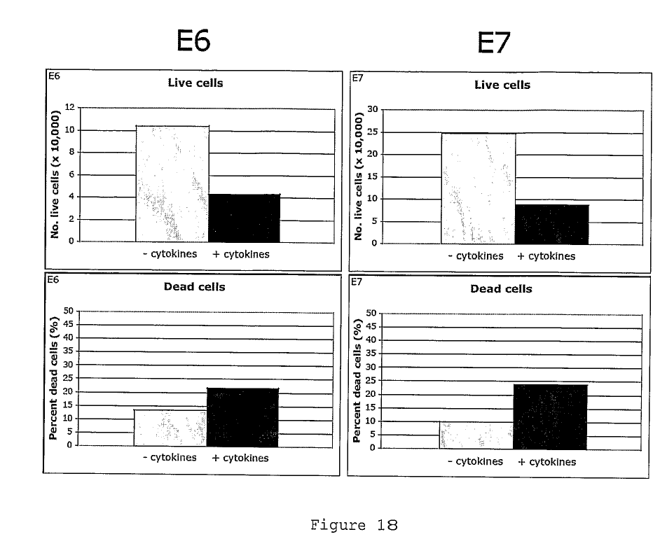

FIG. 18 shows the effect of cytokine treatment for 8 days on primary human fibroblasts (TIG-3) expressing Human Papilloma Virus (HPV) E6 or Human Papilloma Virus (HPV) E7.

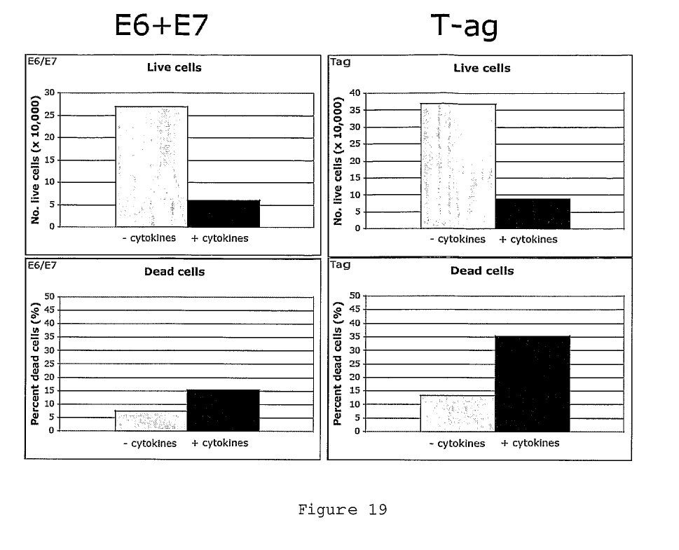

FIG. 19 shows the effect of cytokine treatment for 8 days on primary human fibroblasts (TIG-3) expressing both Human Papilloma Virus (HPV) E6 and Human Papilloma Virus (HPV) E7, or expressing Simian Virus 40 (SV40) large T-antigen.

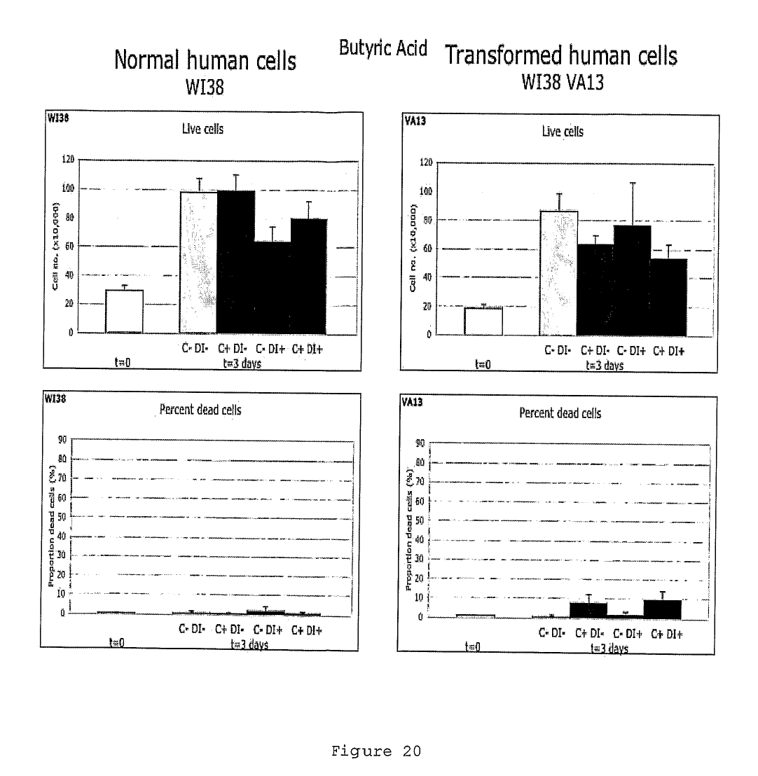

FIG. 20 shows the effect of butyric acid on cytokine treatment of normal WI38 cells and transformed WI38 VA13 cells.

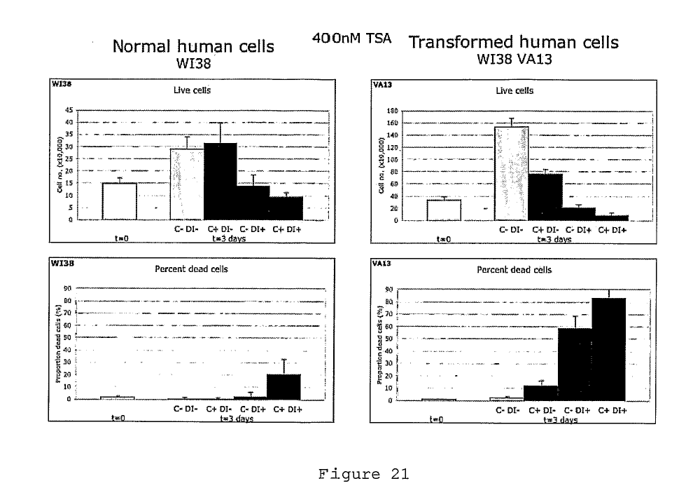

FIG. 21 shows the effect of 400 nM TSA on cytokine treatment of normal WI38 cells and transformed WI38 VA13 cells.

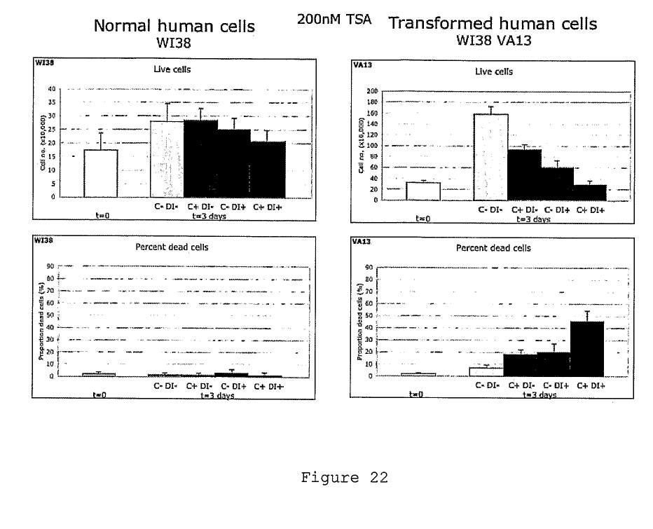

FIG. 22 shows the effect of 200 nM TSA on cytokine treatment of normal WI38 cells and transformed WI38 VA13 cells.

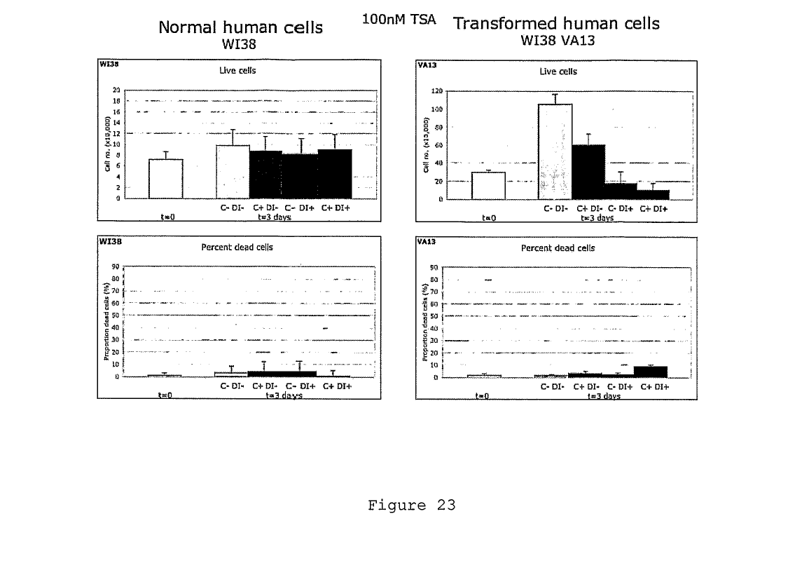

FIG. 23 shows the effect of 100 nM TSA on cytokine treatment of normal WI38 cells and transformed WI38 VA13 cells.

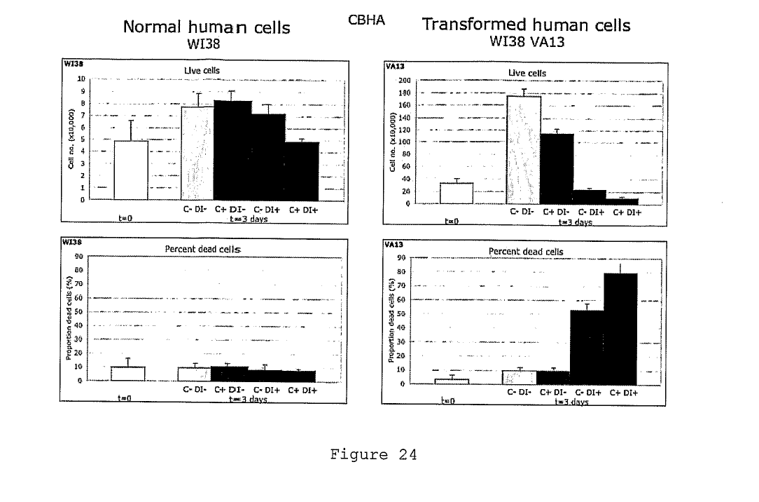

FIG. 24 shows the effect of CBHA on cytokine treatment of normal WI38 cells and transformed WI38 VA13 cells.

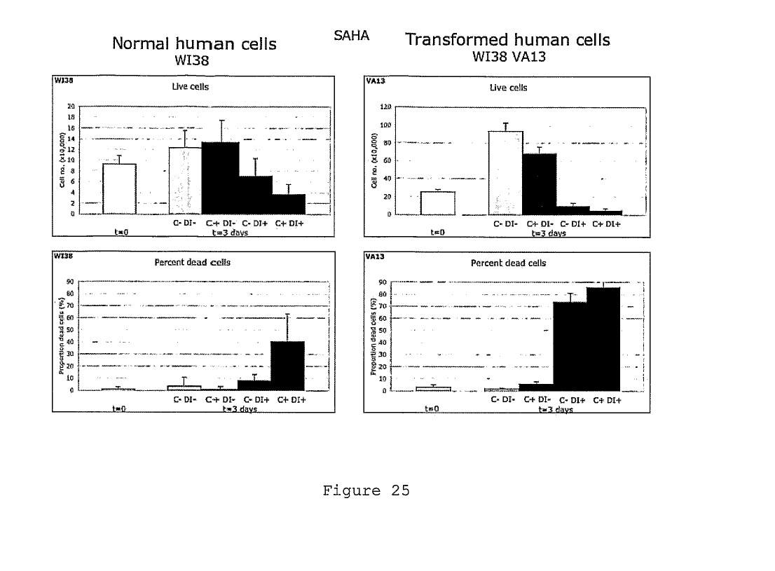

FIG. 25 shows the effect of SAHA on cytokine treatment of normal WI38 cells and transformed WI38 VA13 cells.

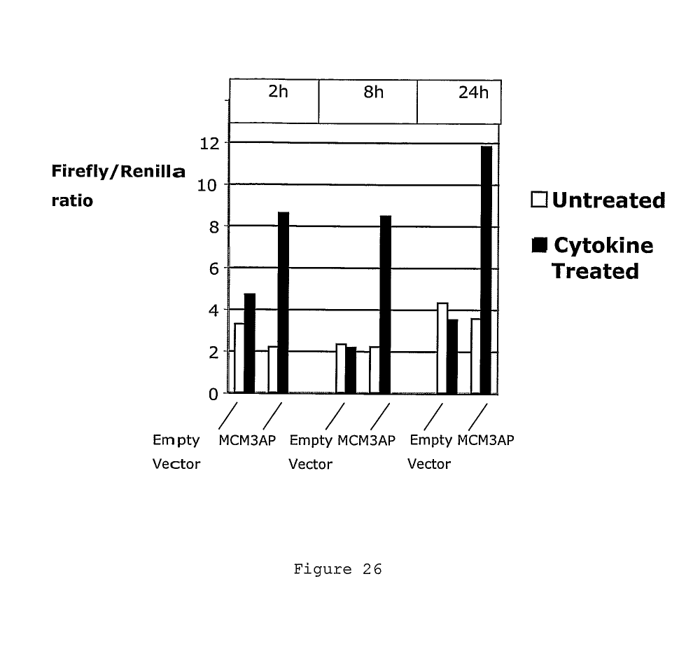

FIG. 26 shows the effect of cytokine treatment on the expression of a luciferase reporter linked to the MCM3AP promoter.

### EXPERIMENTAL

Materials and Methods

DNA and Plasmids

DNA sequence was analysed using Genetyx ver.10 software. Invitrogen FlpIn T-Rex system was used for targeted integration of plasmids, according to manufacturer's instructions.

Poly(A) RNA Purification (for 5′RACE)

Poly(A) RNA was purified from HEK293 cells according to manufacturers instructions (Ambion); briefly cells were lysed and oligo(dT) cellulose was added to the sample and incubated for 1 hour at 20° C., then pelleted. Binding buffer and wash buffer were then added to remove non-specifically bound material and ribosomal RNA. Poly(A)-selected RNA was recovered by incubating the oligo(dT) cellulose pellet in elution buffer at 65° C., and RNA was precipitated with 0.1 volume 5M sodium acetate, 2.5 volumes 100% ethanol, 2 μl glycogen. An RNA pellet was then obtained by centrifugation at 12000 g for 20 minutes at 4° C., which was then washed with 70% ethanol and resuspended in DEPC treated water. Poly(A) RNA was quantitated using a spectrophotometer, and the A₂₆₀/A₂₈₀ ratio was measured which fell between 1.8 and 2.1. Poly(A) RNA was then treated with DNaseI (2 units) (Ambion)

5′ RACE (Rapid Amplification of cDNA Ends)

5 μg Poly(A) RNA was treated with calf intestinal phosphatase (CIP) for 1.5 hours at 50° C. to remove 5′ Phosphate from degraded mRNA, and acid phenol/chloroform extraction was performed to terminate the reaction and remove all protein. RNA was recovered by centrifugation at 12000 g at 4° C., and the pellet was washed with 70% ethanol, and RNA quantitated as before. CIP-treated RNA was treated with tobacco acid pyrophosphatase for 2 hours at 37° C. to remove cap from full-length mRNA (a TAP minus control was also performed). RNA was recovered by phenol/chloroform extraction, and ethanol precipitation, and then quantified. The 5′ RACE adapter was ligated to CIP/TAP treated RNA (along with negative control) using T4 RNA ligase in 1× RNA ligase buffer (Ambion) for 2 hours at 37° C., and RNA was recovered by ethanol precipitation as before. A reverse transcription reaction using oligo dT primer and superscript II reverse transcriptase (Invitrogen) was carried out at 42° C. for 2 hours. The enzyme was inactivated at 70° C., and the reaction mixture was subsequently treated with RNase H1 (Invitrogen). cDNA was purified using Microcon centrifugation devices (Millipore) and used as a template for PCR using various primers.

PCR for 5′RACE:

Initially, 3 rounds of nested PCR were performed: the 1st round with primers homologous to the 5′ PACE adapter sequence and to a region 50 lop downstream of the start codon, with 30 cycles with an annealing temperature of 60° C. The 2nd round with either 25 or 35 cycles of amplification at 60° C. and a different primer homologous to a region 70 bp upstream of the start codon, was performed using a template from the 1st round purified using a PCR purification kit (Qiagen). The 3^{rd} round PCR was performed as per the second round, except the primers used were homologous to a different sequence of the 5′RACE adapter and located 170 bp upstream of the start codon, and the template used was that from the 2nd round.

For subsequent experiments performed to remove background bands, 2 rounds of nested PCR were undertaken, except the 2^{nd} round used a touchdown method, with 5 initial cycles of annealing/extension at 72° C., 5 more at 70° C., followed by 25 cycles at 60-66° C. Samples were run on a 2% agarose gel containing 0.3 ug/ml of ethidium bromide and visualised using a UV luminometer.

Cell Culture and Cell Fractionation

HEK 293 cells (ATCC number CRL-1573) and Hs68 cells (ATCC number CRL-1635) were cultured as monolayers in 10% foetal calf serum in Dulbecco's minimal essential medium at 37° C. in 10% or 5% CO₂ respectively, according to recommendations by the American Tissue Culture Collection. Stable cell lines of HEK 293 cells expressing inducible wild-type or mutant MCM3AP were made by stable transfection of vectors previously described by Takei et al. (2,3). Expression of MCM3AP was induced in these stable cell lines by treatment with doxycycline.

Exponentially growing cells were harvested and fractionated into soluble and structure-bound fractions. Cells were harvested by scraping adherent cells, pelleted by centrifugation at 200 g for 4 minutes and washed in cold PBS. The cell pellet was resuspended in cold hypotonic buffer (10 mM HEPES-KOH pH 7.3, 5 mM KCl, 1.5 mM MgCl₂, 1 mM DTT, 1% NP40 and protease inhibitors) and incubated on ice for 15 minutes. Cells were centrifuged at 10,000 rpm for 5 minutes and the supernatant stored at −80° C. as the soluble protein fraction. The pellet was washed in hypotonic buffer, resuspended in hypotonic buffer containing 0.5M NaCl and incubated on ice for 15 minutes. The suspension was centrifuged at 10,000 rpm for 5 minutes and the supernatant stored at −80° C. as the structure-bound fraction.

Samples were separated by 7.5% SDS-PAGE and transferred to PVDF membrane at a constant voltage of 10V for 1 hour. The membrane was blocked with 5% non-fat milk in PBS and probed with antibody in 5% non-fat milk in PBS. The bound antibodies were detected with peroxidase-conjugated secondary antibody (Amersham Biosciences) and protein bands visualised by the colorimetric method using ECL or ECLplus (Amersham Biosciences). Primary antibodies used were ‘253’ rabbit polyclonal antibody to MCM3AP (1) which detects both MCM3AP and GANP, and ‘sc-9850 N-19’ goat polyclonal antibody to MCM3 (Santa Cruz).

Human cells WI38 (ATCC number CCL-75), WI38 VA13 (ECACC number 85062512), SaOS2 (ECACC number 89050205), U20S (ATCC number HTB-96), HeLa (CCL-2), HEK 293 (ATCC number CRL-1573) and Hs68 (ATCC number CRL-1635) were obtained from ATCC, ECACC or CR-UK cell services. All cells were cultured as monolayers in 10% foetal bovine serum in Dulbecco's minimal essential medium at 37° C. in 5% CO₂, except SaOS2 cells which were cultured in McCoy's 5a medium supplemented with 1.5 mM L-glutamine and 15% foetal bovine serum. VA13 cells are WI38 primary fibroblasts transformed with SV40 virus and therefore share the same genetic background.

Human cells TIG-3 cells (Ohashi et al. 1980 Exp Gerontol. 1980; 15(2): 121-133) expressing the ectotropic receptor were retrovirally infected with empty pBABEpuro vector or the same vector containing genes encoding HPV E6, HPV E7, both HPV E6 and HPV E7 or SV40 T-antigen and selected in puromycin. Cells were cultured as monolayers in 10% fcetal bovine serum in Dulbecco's minimal essential medium at 37° C. in 5% CO₂.

Cytokine Treatment

Cells were treated with Interleukin 6 (20 ng/ml) for 24 hours, followed by addition of antibody 80M2 (1 μg/ml), which modulates TNFα receptor 2, and TNFα (10 ng/ml). TNFα was added to the cell culture medium 30 minutes after addition of 80M2. All agents were added to the cell culture medium in liquid form. Cells were harvested after the indicated number of days of treatment, where ‘time zero’ corresponds to the point of addition of Interleukin 6.

Cell Number, Cell Viability and Apoptosis Assays

Cells were assayed for apoptosis by Annexin V staining (Oncogene, Annexin V-FITC or Annexin V-Biotin apoptosis detection kit) and TUNEL assay (BD Biosciences, ApoAlert DNA fragmentation assay kit). Cells were assayed for Annexin V and TUNEL assay by flow cytometry, according to manufacturer's instructions. Attached cells were harvested and counted using a haemocytometer to give total attached cell number.

To measure cell viability and total cell number, cells in suspension and adherent cells were collated and analysed by resuspending with a known concentration of CaliBRITE unlabelled beads (Becton Dickinson) plus propidium iodide and counting the number of live and dead cells per 3000 beads using a FACSCalibur flow cytometer (Becton Dickinson). Alternatively, an automated trypan blue assay using a Vi-Cell XR Cell Viability Analyzer (Beckman Coulter) was employed.

Results were confirmed by incubating cells in 0.04% Trypan Blue (SIGMA) for 10 minutes and counting Trypan Blue negative and positive cells using a haemacytometer.

Cytokine and Deacetylase Inhibitor Treatment

Cells were treated with Interleukin 6 (20 ng/ml) for 24 hours, followed by addition of antibody 80M2 (1 μg/ml), which modulates TNFα receptor 2, and TNFα (10 ng/ml). TNFα was added to the cell culture medium 30 minutes after addition of 80M2. Deacetylase inhibitor was added 30 minutes after TNFα at the following concentrations: 5 mM butyric acid; 100 nM, 200 nM or 400 nM TSA; 2.5 μg/ml CBHA; 5 μg/ml SAHA.

CBHA and SAHA were obtained from Aton Pharmaceuticals Ltd.

All agents were added to the cell culture medium in liquid form. For cytokine treatment alone, cells were harvested and counted 48 or 72 hours after addition of TNFα, as indicated in Table 1, corresponding to a total of 3 or 4 days treatment respectively. For cytokine treatment in combination with deacetylase inhibitors, cells were harvested and counted 48 hours after addition of deacetylase inhibitor.

Immunofluorescence

Equal numbers of WI38 and VA13 cells were seeded into 60 mm tissue culture dishes containing coverslips and treated with or without cytokines for four days as described above. At the end of the treatment, coverslips were removed, washed gently in PBS and fixed in 3.7% formaldehyde (SIGMA) at room temperature for 10 minutes. Coverslips were then washed with PBS and stored at −20° C. in 80% ethanol.

To stain the cells, coverslips were washed in PBS and blocked in 5% BSA blocking solution for 30-60 minutes at room temperature. Coverslips were incubated in primary antibody for 1 hour at 37° C. or overnight at 4° C., washed three times in blocking solution, incubated in secondary antibody for 45-60 minutes at 37° C. and washed three times in blocking solution. Coverslips were washed in PBS, rinsed in water and mounted on slides with Vectashield anti-fade solution containing DAPI.

Primary antibodies used were: SV40 T-antigen Pab 340 monoclonal antibody (Cancer Research UK) 1:100 dilution; cleaved caspase 3 Asp175 rabbit polyclonal antibody (Cell Signaling Technology) 1:50 dilution. Secondary antibodies used were: sheep anti-mouse Ig, fluorescein linked whole antibody (Amersham) 1:100 dilution; donkey anti-rabbit Texas Red (Amersham) 1:100 dilution.

Dual-Luciferase Assay

HEK 293 cells were split into 6 well plates at a density of 50 000 cells/well and the next morning, were pre-treated with IL6 (20 ng/ml) for 24 hours. Cells were treated with 80M2 monoclonal antibody (1 μg/ml) for 30 mins, then with TNFalpha (10 ng/ml) for the indicated periods of time (2-24 hours), and co-transfected with pGL3 luciferase plasmid with wild-type MCM3AP promoter sequence and a constant amount of renilla luciferase control plasmid, pRL-TK, using Polyfect transfection reagent (Qiagen) Cells were harvested 50 hours post-addition TNFalpha and assayed for luciferase activity (Promega). Briefly, each well was lysed with 250 μl of passive lysis buffer (Promega), and subjected to 2 freeze-thaw cycles. 10 μl of lysate was then added to 100 μl of luciferase assay reagent (Promega) and placed into a luminometer (Turner Systems) and firefly luciferase reading initiated. 100 μl of stop and glo reagent (Promega) was then added, vortexed and renilla luciferase reading initiated. The firefly/renilla luciferase ratio was calculated.

Results

MCM3AP Introns and Exons

The exons of GANP have recently been renumbered. A new exon that had been found between exon 15 and exon 16 of GANP was previous designated ‘exon 15.5’, but since then it has been renamed ‘exon 16’ and all subsequent exons and introns renumbered. FIG. 1b shows the schematic of MCM3AP promoter in intron 16 of GANP with the new numbering system.

Identification of the MCM3AP Promoter

DNA sequence homology and amino acid identity between the C-terminal of GANP and MCM3AP were established upon isolation of GANP cDNA from mouse and human cDNA libraries (4, 7). The transcriptional start site of MCM3AP is contained in exon 17 (previously designated exon 16) of GANP in the region of sequence identity, such that exons 17 to 29 of GANP (previously designated exons 16 to 28) are translated in the same reading frame as exons 1 to 13 of MCM3AP, producing proteins with amino acid identity (FIG. 1b). Thus, the amino acid sequence of the 82 kilodalton MCM3AP protein is identical to that of the carboxy terminal region of the 210 kilodalton GANP protein (FIG. 1a).

Using 5′ RACE (Rapid Amplification of cDNA Ends) and oligonucleotide capping, the promoter region of MCM3AP was mapped to intron 16 (formerly designated intron 15) of GANP and the transcription initiation site located adjacent to it at the start of MCM3AP exon 1 (FIG. 1b and FIG. 2). Transcriptional activation from the MCM3AP promoter has been confirmed by CAT assay. Consensus sequence binding sites were identified in the promoter region of MCM3AP for transcription factors NF-IL6, AP1, NF-κB and p53 (FIG. 1b and FIG. 2). Note that the GANP promoter does not contain these, or similar, binding sites. Thus MCM3AP has its own specific promoter which contains binding elements for transcription factors which are not found in the GANP promoter.

The MCM3AP promoter is activated by cytokines Three of the transcription factors with binding elements in the MCM3AP promoter, namely NF-κB, NF-IL6 and AP-1, are known to be activated by the cytokines TNFα, interleukin-6 (IL6) and TNFα again respectively.

HEK 293 cells (ATCC number CRL-1573) were treated with Interleukin 6 (20 ng/ml), followed 24 hours later by TNFα (10 ng/ml) and antibody 80M2 (1 μg/ml) which modulates TNFα receptor 2. Western blots are shown in FIG. 3 recording the expression of GANP, MCM3AP and MCM3 over the following three days following cytokine treatment. MCM3AP protein, but not GANP protein, is induced synergistically in HEK 293 cells by the combination of IL6, TNFα and an antibody against TNFα receptor II, 80M2. Treatment with IL6 or TNFα alone does not stimulate MCM3AP significantly. GANP expression is not responsive to cytokine treatment.

Cytokine Treatment Induces Apoptosis in HEK 293 Cells

We examined the cell fate outcome of upregulating MCM3AP by cytokine treatment in HEK 293 cells. Treatment of HEK 293 cells with the same combination of cytokines that upregulates MCM3AP induces cell death by apoptosis (FIG. 4). Cells were treated with IL6 for 24 hours, followed by addition of TNFα and antibody 80M2. Attached cells were assayed for apoptosis and total cell number 24 hours and 36 hours later respectively. The combination of IL6, TNFα and 80M2 leads to extensive induction of apoptosis and a decrease in cell number over time. Note that the cytokines exhibit synergy in cell killing as well as induction of MCM3AP, as the induction of apoptosis by TNFα without IL6 is markedly reduced compared to apoptosis induced by TNFα and IL6 (FIG. 4).

Overexpression of MCM3AP Augments Cell Killing by TNFα

HEK 293 cells were stably transfected with a construct encoding doxycycline-inducible MCM3AP. The expression level of MCM3AP upon induction with doxycyline was comparable with the level of MCM3AP induced by cytokine treatment. Cells were treated with or without doxycycline for 12 hours and then incubated with TNFα plus 80M2 for the times indicated. 80M2 is a TNF2 receptor antibody that modulates the receptor and boosts downstream signaling when TNF alpha binds TNF receptor 2. Cells were harvested and assayed for attached cell number and apoptosis, both by annexin-V staining and TUNEL assay (FIG. 5).

Over-expression of MCM3AP by doxycycline augmented cell death induced by TNFα plus 80M2 in all assays. Note that this experiment was conducted in the absence of IL6, which indicates that induction of MCM3AP by doxycycline can substitute for IL6 in the synergistic effect on cell killing with TNFα (FIG. 5 and compare with FIG. 4). Thus, induction of MCM3AP expression with doxycycline sensitises HEK 293 cells to cell death in response to TNFα and antibody 80M2, even in the absence of interleukin 6.

To establish the functional contribution of MCM3AP to cell killing, doxycycline-inducible mutants of MCM3AP deficient in either binding to MCM3 or acetylase activity (3) were stably transfected into HEK 293 cells. Cells were treated with or without doxycycline and with or without TNFα plus 80M2 for 36 hours and cell number determined before and after treatment (FIG. 6). Cells expressing MCM3AP mutants deficient in acetylase activity or MCM3 binding showed no significant reduction in cell number compared to controls. Significant reduction in cell number was observed only in cells expressing wild-type MCM3AP treated with doxycycline and TNFα plus 80M2 for 36 hours. This demonstrates that cell killing by cytokines is dependent on both the acetylase activity and MCM3 binding of MCM3AP since mutations that abolish either acetylase activity or MCM3 binding relieve induction of cell death by TNFα.

Cytokine-Induced Cell Killing is Selective

HEK 293 cells are virally transformed human kidney cells that are p53 and Rb compromised and capable of forming tumours in nude mice. The responses of tumorigenic cells and normal diploid cells to cytokine treatment were compared. Normal human diploid fibroblasts, Hs68 cells, and HEK 293 cells were treated with the same combination of cytokines used to induce MCM3AP in HEK 293 cells, namely incubation with IL6 for 24 hours followed by addition of TNFα and 80M2 (see FIG. 3). This treatment up regulated MCM3AP in Hs68 cells over time, as in HEK 293 cells albeit to a lesser extent. Cells were harvested after a further 48 hours (HEK 293) or 72 hours (Hs68), according to cell cycle time. Cells in suspension and adherent cells were collated and incubated with Trypan Blue in a dye exclusion cell viability assay. Trypan Blue negative and positive cells were counted and plotted as live and dead cells respectively (FIG. 7).

Cytokine treatment was observed to cause a marked decrease in live cell number of HEK 293 cells versus the slight increase in live cell number of Hs68 cells compared to controls. Consistent with this, cytokine treatment caused a great increase in the proportion of dead HEK 293 cells but failed to cause a significant increase in cell death of normal human fibroblast Hs68 cells. Thus cytokine treatments that cause apoptotic cell death in HEK 293 cells do not kill normal human fibroblasts (Hs68 cells).

The ability of cytokines to selectively kill cells was further investigated in primary, virally transformed and tumour-derived cells of various tissue origins. The results are set out in Table 1 and FIGS. 8 to 13.

Cytokine treatment for 3-4 days was observed to have little effect on primary human cells of two different cell types. In the presence of cytokines, live cells continued to proliferate, indicating no cytostatic effect and the cytokine treated cells showed a slight growth advantage.

Two transformed and three tumour-derived tumourigenic cell types were tested. Cytokine treatment over 3-4 days was observed to be lethal for both virally transformed and tumour-derived cells, which showed a significant decrease in live cell number. Cells that survived treatment continued to proliferate.

Cytokine treatment was found to be lethal for tumorigenic cells of both fibroblast and epithelial origin, indicating that sensitivity to killing by cytokine treatment was independent of tissue origin.

Of the seven cell types were tested, two had intact Rb and p53 checkpoints and five had impaired or deficient Rb and p53 checkpoints. These correspond to primary and tumorigenic cells respectively. Cells with impaired Rb and p53 checkpoints were observed to be sensitive to cytokine treatment whereas cells with intact Rb and p53 checkpoints were insensitive.

Cytokine treatment was therefore shown to be lethal to virally transformed and tumour-derived cells, regardless of tissue origin, but to have no significant effect on primary cells.

VA13 cells are WI38 primary fibroblasts transformed with SV40 virus and therefore share the same genetic background. To illustrate the selectivity of the effect of cytokine treatment, mixed cultures of equal numbers of WI38 and VA13 cells were seeded and treated with the cytokines for four days. Cells were visualised using the DNA stain, DAPI, to identify all cells and an antibody against SV40 large T antigen to identify VA13 cells. The colocalisation of DAPI plus SV40 T antigen could therefore be observed.

After four days without cytokine treatment, VA13 cells (T antigen positive) were found to have outgrown WI38 cells, due to their faster cell cycle time. However, in the presence of cytokines, few VA13 cells remain after four days, whilst the number of WI38 cells is unchanged compared to the untreated control. Cell viability data provides indication that the VA13 cells in the mixed culture have died. Additional staining of the mixed cultures with an antibody against cleaved Caspase 3 (an early apoptosis marker) shows that, of those VA13 cells remaining after cytokine treatment, a number are already undergoing apoptosis.

Human Papilloma Virus (HPV) E6 targets and inactivates p53 protein, HPV E7 targets and inactivates Rb protein and Simian Virus 40 (SV40) large T-antigen targets and inactivates both p53 and Rb proteins. Primary human fibroblasts, TIG-3 cells, expressing these viral oncoproteins were treated with or without cytokines (as described above) and the number of live and dead cells after treatment was measured.

FIGS. 14 to 16 show the number of live and dead cells observed after four days of cytokine treatment. Control cells are TIG-3 cells expressing the ecotropic receptor (ER) and TIG-3 cells expressing the empty pBABEpuro vector (top). Test cells are TIG-3 cells expressing the pBABEpuro vector containing genes encoding HPV E6, HPV E7, both HPV E6 and E7 or SV40 large T-antigen (T-ag) as indicated. Test cells which have inactivated p53 (those expressing E6, E6+E7 or T-ag) have reduced live cell number and increased proportion of dead cells after cytokine treatment, whereas test cells which have inactivated Rb alone (those expressing E7) are unaltered compared to controls.

FIGS. 17 to 19 show that after eight days of cytokine treatment the number of live cells in all test cell strains is further diminished compared to untreated controls, and that cells which have inactivated Rb (those expressing E7) have been comparably affected by cytokine treatment to those which have inactivated p53, alone or in combination with Rb.

These results show that an impaired p53 checkpoint is the primary determinant of selective cell killing, and that an impaired Rb checkpoint enhances the effect at later timepoints once selective killing is underway.

The Synergistic Effect of Cytokine Treatment and Deacetylase Inhibitors

The effect of the commercially available deacetylase inhibitors, trichostatin A (TSA) and butyric acid, both alone and in combination with cytokine treatment was observed on transformed and normal cells. The results are shown in FIGS. 20 to 23.

The deacetylase inhibitors tested showed a variety of efficacies in cell killing, butyric acid being the least effective. TSA was found to be efficient in selectively killing transformed cells with minimal effect on WI38 cells. This experiment was confirmed by repetition, and showed that three days of cytokine treatment in combination with 400 nM TSA killed the vast majority (80-90%) of transformed cells. However, it also induced ˜20% cell death in normal cells in combination with cytokine treatment and lead to a reduction in live cell number over time (compare with t=0 value). Lower concentrations of TSA were investigated and the effect of TSA was found to be concentration dependent.

An optimum concentration between 200 nM and 400 nM may maximise killing of transformed VA13 cells and minimise deleterious cytostatic or killing effects on normal WI38 cells.

The effect of the deacetylase inhibitors cinnamic acid bishydroxamic acid (m-carboxycinnamic acid bishydroxamide: CBHA) and suberoylanilide hydroxamic acid (SAHA) were examined on normal and transformed cells alone and in combination with cytokine treatment. These experiments were carried out according to the same protocol used for TSA and butyric acid and were confirmed by repetition. CBHA was used at a concentration of 2.5 μg/ml and SAHA at 5 μg/ml. The results are shown in FIGS. 24 and 25. CBHA and SAHA showed similar efficacy in selective cell killing compared to 400 nM TSA. Three days of cytokine treatment in combination with CBHA or SAHA killed the vast majority (80-90%) of transformed cells. CBHA did not kill primary WI38 cells above background levels. SAHA was very effective in killing VA13 cells alone and in combination with cytokines but caused significant cell death (˜40%) of normal WI38 cells when used in combination with cytokine treatment.

The role of the MCM3AP promoter in the regulation of MCM3AP MCM3AP has been shown to be upregulated at the protein level in response to cytokine treatment. In order to determine if MCM3AP is also induced at the transcriptional level and hence contains a cytokine-responsive promoter element, a dual-luciferase assay was performed.

HEK 293 cells were pre-treated with IL6 for 24 hours, then treated with 80M2 monoclonal antibody for 30 minutes and then treated with TNFalpha. At time points either 2, 8 or 24 hours post-addition of TNFalpha, cells were co-transfected with a pGL3 firefly luciferase plasmid fused to the promoter sequence of MCM3AP, and a pRL-TK renilla luciferase plasmid (containing a weak promoter sequence) and harvested 50 hours post-addition of TNFalpha. As a control, an empty pGL3 plasmid was also co-transfected. FIG. 26 shows an approximate four-fold increase in the cytokine treated samples transfected with pGL3-MCM3AP compared to the untreated samples, at every timepoint tested. This increase is not observed in the samples transfected with empty vector, indicating that the increase is due to the presence of the MCM3AP promoter sequence, and not an artefact of cytokine treatment on cells. This provides indication that MCM3AP is cytokine-responsive at the transcriptional level.

Luciferase values in untreated cells transfected with pGL3-MCM3AP are relatively unchanged from cells transfected with empty pGL3 vector and only increase after cytokine treatment. This correlates with the observation that MCM3AP is expressed at a low level in the cell and is upregulated after cytokine treatment. The present inventors have shown that MCM3AP is encoded within the sequence of the GANP gene, but is independently regulated by its own promoter. The MCM3AP promoter lies within intron 16 (formerly designated intron 15) of GANP and contains binding sites for several transcription factors which are not found in the promoter of GANP. These binding sites, namely NF-IL6, AP1, NF-κB and p53, allow independent regulation of MCM3AP in response to signalling pathways in vivo.

The cytokines TNFα and interleukin-6 (IL6) activate the transcription factors NF-κB and NF-IL6 respectively. Treatment of HEK 293 cells with combinations of these cytokines and the TNFRII antibody, 80M2, induces up-regulation of MCM3AP with concomitant and substantial apoptotic cell death. There is no up-regulation of expression of GANP. Over-expression of MCM3AP can substitute for IL6 in the augmentation of cell killing by TNFα, indicating that up-regulation of MCM3AP has a causal role in cell death induced by cytokines. Furthermore, the acetyltransferase activity and MCM3 binding activity of MCM3AP are required for its role in cell killing, as mutants of MCM3AP deficient in either of these activities fail to augment cell killing by TNFα.

Importantly, the cytokine treatment that up-regulates MCM3AP and induces cell death in tumour cell lines, irrespective of the tissue of origin, does not induce significant cell death in normal human cells. Our data indicate that this is not due to a failure to up regulate MCM3AP protein.

Synergistic combinations of cytokines as described here, or mimics thereof, may therefore be useful therapeutically, optionally in combination with deacetylase inhibitors, to upregulate MCM3AP in cancer cells and induce cell death.

TABLE 1

| | | Transformation | | Rb | | p53 | No. | ↓ live | ↑ dead |
|---|---|---|---|---|---|---|---|---|---|
| | Cell type | status | Rb | checkpoint | p53 | checkpoint | days | cells? | cells? |
| Hs68 | Fibroblast | Primary | WT | Yes | WT | Yes | 4 | No | No |
| WI38 | Fibroblast | Primary | WT | Yes | WT | Yes | 3 | No | No |
| | | | | | | | 4 | No | No |
| VA13 | Fibroblast | Virally | WT | No | WT | No | 3 | Yes | No |
| | | transformed | | SV40 T-Ag | | SV40 T-Ag | 4 | Yes | Yes |
| HEK | Epithelial | Virally | WT | No | WT | No | 3 | Yes | Yes |
| 293 | | transformed | | Ad 5 | | Ad 5 | 4 | Yes | Yes |
| HeLa | Epithelial | Tumour- | WT | No | WT | No | 3 | Yes | Yes |
| | | derived | | HPV E6 | | HPV E7 | 4 | Yes | Yes |
| U2OS | Fibroblast- | Tumour- | WT | No | WT | No | 4 | Yes | Yes |
| | derived | derived | | Me'd p16 pr | | Me'd ARF pr | 4 | Yes | Yes |
| SaOS2 | Fibroblast- | Tumour- | Mutant | No | Mutant | No | 4 | Yes | Yes |
| | derived | derived | | non-fun'l Rb | | p53 deletion | 4 | Yes | Yes |

### REFERENCES

1. Takei, Y. & Tsujimoto, G. (1998) J Biol Chem 273, 22177-80.

2. Takei, Y. et al (2001) EMBO Rep 2, 119-23.

3. Takei, Y. et al. (2002) J Biol Chem 277, 43121-5.

4. Abe, E. et al. (2000) Gene 255, 219-27.

5. Kuwahara, K. et al. (2001) Proc Natl Acad Sci U S A 98, 10279-83.

6. Kuwahara, K. et al. (2004) Proc Natl Acad Sci U S A 101, 101 C-5.

7. Kuwahara, K. et al. (2000) Blood 95, 2321-8.

8. Kono, Y. et al. (2002) Genes Cells 7, 821-34.

1. A method of killing a cancer cell in an individual having cancer condition comprising up-regulating the expression of MCM3AP in said cell.

2. The method according to claim 1 wherein said expression induces selective lethality in cancer cells relative to non-cancer cells.

3. The method according to claim 1 wherein expression of MCM3AP is up regulated by increasing the amount and/or activity of both TNFα and IL-6 transcription factors in said cell.

4. The method according to claim 3 wherein the level and/or activity of TNFα associated transcription factors is increased by one or more of: exposing the cell to increased amounts of a TNFα cytokine, increasing the activity of a TNFα cytokine to which the cell is exposed and exposing the cell to a TNFα analogue or mimetic.

5. The method according to claim 3 wherein the level and/or activity of IL-6 associated transcription factors is increased by one or more of: exposing the cell to increased amounts of a IL-6 cytokine, increasing the activity of a IL-6 cytokine to which the cell is exposed and exposing the cell to a IL-6 analogue or mimetic.

6. The method according to claim 3 wherein the level and/or activity of TNFα associated transcription factors in the cell is increased by administering to the individual a TNFα cytokine, a nucleic acid which encodes a TNFα cytokine, a recombinant cell which expresses a TNFα cytokine, a TNFα inducer which increases the exposure of said cell to a TNFα cytokine, a TNFα potentiator which increases the activity of a TNFα cytokine, or a combination of two or more thereof.

7.-10. (canceled)

11. The method according to claim 10 wherein the TNFα potentiator is an antibody that specifically binds to a TNFα receptor.

12. A method according to claim 3 wherein the level and/or activity of IL-6 associated transcription factors in the cell is increased by administering to the individual an IL-6 cytokine, a nucleic acid which encodes a IL-6 cytokine, recombinant cell which expresses an IL-6 cytokine, an IL-6 inducer, an IL-6 potentiator which increases the activity of a IL-6 cytokine, or a combination of two or more thereof.

13.-16. (canceled)

17. The method according to claim 1 comprising contacting the cancer cell with a deacetylase inhibitor.

18. The method according to claim 17 wherein the deacetylase inhibitor is administered to the individual.

19. The method according to claim 17 wherein the deacetylase inhibitor is selected from the group consisting of hydroxamate, cyclic peptide, aliphatic acid, benzamide and electrophilic ketone.

20. The method according to claim 17 wherein the deacetylase inhibitor is selected from the group consisting of CBHA, SAHA, TSA and butyric acid.

21. The method according to claim 1 wherein said cancer cell is deficient in p53 expression and/or activity.

22. The method according to claim 1 wherein said cancer cell is deficient in pRb expression and/or activity.

23.-31. (canceled)

32. A method of treating cancer in a subject in need thereof, comprising providing to the subject a composition comprising a TNFα factor and an IL-6 factor, thereby treating the cancer.

33. The method according to claim 32 wherein the cancer is a p53 deficient cancer.

34. The method according to claim 32 wherein the cancer is a pRb deficient cancer.

35. The method according to claim 32 wherein;
the TNFα factor is selected from the group consisting of: a TNFα cytokine, a nucleic acid encoding a TNFα cytokine, a recombinant cell expressing a TNFα cytokine, a TNFα potentiator, a TNFα inducer and a TNFα analogue or mimetic, and wherein;
the IL-6 factor is selected from the group consisting of: a IL-6 cytokine, a nucleic acid encoding an IL-6 cytokine, a recombinant cell expressing an IL-6 cytokine, an IL-6 potentiator, an IL-6 inducer and an IL-6 analogue or mimetic.

36. The method according to claim 32 wherein said medicament further comprises a deacetylase inhibitor.

37. The method according to claim 36 wherein the deacetylase inhibitor is selected from the group consisting of hydroxamate, cyclic peptide, aliphatic acid, benzamide and electrophilic ketone.

38. The method according to claim 37 wherein the deacetylase inhibitor is selected from the group consisting of CBHA, SAHA, TSA and butyric acid.

39. A pharmaceutical composition comprising a TNFα factor and an IL-6 factor and a pharmaceutically acceptable excipient.

40. A composition according to claim 39 wherein;
the TNFα factor is selected from the group consisting of: a TNFα cytokine, a nucleic acid encoding a TNFα cytokine, a recombinant cell expressing a TNFα cytokine, a TNFα potentiator, a TNFα inducer and a TNFα analogue or mimetic, and wherein;
the IL-6 factor is selected from the group consisting of: a IL-6 cytokine, a nucleic acid encoding an IL-6 cytokine, a recombinant cell expressing an IL-6 cytokine, an IL-6 potentiator, an IL-6 inducer and an IL-6 analogue or mimetic

41. The composition according to claim 39 wherein said composition further comprises a deacetylase inhibitor.

42. The composition according to claim 41 wherein the deacetylase inhibitor is selected from the group consisting of hydroxamate, cyclic peptide, aliphatic acid, benzamide and electrophilic ketone.

43. The composition according to claim 42 wherein the deacetylase inhibitor is selected from the group consisting of CBHA, SAHA, TSA and butyric acid.

44. A method of making a pharmaceutical composition comprising admixing a TNFα factor and an IL-6 factor with a pharmaceutically acceptable excipient.

45. The method according to claim 44 further comprising admixing a deacetylase inhibitor with said TNFα factor, IL-6 factor and excipient.

46. The method according to claim 45 wherein the deacetylase inhibitor is selected from the group consisting of hydroxamate, cyclic peptide, aliphatic acid, benzamide and electrophilic ketone.

47. The method according to claim 46 wherein the deacetylase inhibitor is selected from the group consisting of CBHA, SAHA, TSA and butyric acid.

48. A screening method for an anti-cancer agent comprising;
providing a nucleic acid construct comprising a MCM3AP promoter operably linked to a reporter gene,
contacting the construct with a test compound and;
determining the expression of the reporter gene.

49. The screening method according to claim 48 wherein the MCM3AP promoter has a sequence which shares at least 50% sequence identity with a sequence shown in FIG. 2.

50. The screening method according to claim 49 wherein the MCM3AP promoter has the sequence shown in FIG. 2.

51. The screening method according to claim 48 wherein the nucleic acid construct is comprised within a host cell.

52. The screening method according to claim 48 wherein the construct is contacted with the test compound in the presence of IL-6.

53. The screening method according to claim 48 wherein the construct is contacted with the test compound in the presence of TNFα.

54. The screening method according to claim 48 comprising identifying the test compound as a candidate anti-cancer agent.

55. The screening method according to claim 54 comprising formulating the test compound with a pharmaceutically acceptable excipient.

56. A nucleic acid construct comprising a MCM3AP promoter operably linked to a reporter gene.

57. The nucleic acid construct according to claim 56 wherein the MCM3AP promoter has a sequence having at least 50% sequence identity to the sequence shown in FIG. 2.

58. The nucleic acid construct according to claim 57 wherein the MCM3AP promoter has a sequence shown in FIG. 2.

59. An expression vector comprising the nucleic acid construct according to claim 56.

60. A host cell comprising an expression vector according to claim 59.

61. The host cell according to claim 60 that is a mammalian cell.
